(19) United States
(12) Patent Application Publication
(10) Pub. No.: US 2025/0035611 A1
(43) Pub. Date: Jan. 30, 2025

(54) BREATH SENSOR, GAS SENSOR, AND BREATH SENSING METHOD

(71) Applicant: Asahi Kasei Microdevices Corporation, Tokyo (JP)

(72) Inventors: Yuta TAKAGI, Tokyo (JP); Naoya UGOMORI, Tokyo (JP); Tetsuro OGATA, Tokyo (JP); Seiryu SASAKI, Tokyo (JP); Takaaki FURUYA, Tokyo (JP); Shota ISSHIKI, Tokyo (JP)

(21) Appl. No.: 18/780,545

(22) Filed: Jul. 23, 2024

(30) Foreign Application Priority Data
Jul. 27, 2023 (JP) 2023-122831
Jul. 27, 2023 (JP) 2023-122832
Jun. 24, 2024 (JP) 2024-101204

(51) Int. Cl. G01N 33/497; A61M 16/00

(52) U.S. Cl. CPC G01N 33/497; A61M 2016/0024

(57) ABSTRACT

Provided is a breath sensor which senses a breath generated by breathing, including: a light emitting unit which emits light toward a path through which the breath passes; a first light receiving unit which receives at least a part of the light emitted from the light emitting unit and outputs a first light reception signal according to a light reception result; and an operating unit which performs an operation on the first light reception signal, wherein the operating unit calculates a baseline of a waveform of based on a frequency component of the first light reception signal lower than a first cut-off frequency that is set, and senses the breath based on a signal obtained by removing the baseline from the first light reception signal.

The contents of the following patent application(s) are incorporated herein by reference:
NO. 2023-122831 filed in JP on Jul. 27, 2023
NO. 2023-122832 filed in JP on Jul. 27, 2023
NO. 2024-101204 filed in JP on Jun. 24, 2024.

### BACKGROUND

### 1. Technical Field

The present invention relates to a breath sensor, a gas sensor, and a breath sensing method.

### 2. Related Art

Conventionally known is a measuring device which measures a breathing gas, a concentration of a gas, or a respiratory rate, as shown in Patent Documents 1 to 3 for example.

### PATENT DOCUMENTS

Patent Document 1: Japanese Patent No. 5351583
Patent Document 2: Japanese Patent Application Publication No. 2022-3321
Patent Document 3: Japanese Patent No. 3627243

### BRIEF DESCRIPTION OF THE DRAWINGS

FIG. 1 is a diagram illustrating an example of a breath sensor 10 according to one embodiment of the present invention.

FIG. 2 is a diagram illustrating an example of a signal processing.

FIG. 3 is a diagram illustrating an example of a baseline calculation.

FIG. 4A is a diagram illustrating an example of a result of using a constant first cut-off frequency in the baseline calculation.

FIG. 4B is a diagram illustrating an example of a determination signal obtained by calculating a difference between a light reception signal ratio and a baseline calculation value from FIG. 4A.

FIG. 5A is a diagram illustrating an example of a result of changing the first cut-off frequency in accordance with a magnitude relationship relative to a signal value at an immediately preceding timing in the baseline calculation.

FIG. 5B is a diagram illustrating an example of a determination signal calculated from the light reception signal ratio and the baseline illustrated in FIG. 5A.

FIG. 6A is a diagram illustrating an example of the signal processing using equal cut-off frequencies respectively for the first light reception signal and a second light reception signal.

FIG. 6B is a diagram illustrating an example of the light reception signal ratio calculated from the first light reception signal and the second light reception signal illustrated in FIG. 6A.

FIG. 7A is a diagram illustrating an example of a signal processing using different cut-off frequencies respectively for the first light reception signal and the second light reception signal.

FIG. 7B is a diagram illustrating an example of a ratio between the first light reception signal and the second light reception signal in FIG. 7A.

FIG. 8 is a diagram illustrating another example of the breath sensor 10 according to one embodiment of the present invention.

FIG. 9A is a diagram illustrating an example configuration of a light emitting unit 16.

FIG. 9B is a diagram illustrating an example of a gas sensor 11 according to one embodiment of the present invention.

FIG. 10 is a flowchart illustrating an example of a breath sensing method according to one embodiment of the present invention.

FIG. 11 is a flowchart illustrating another example of the breath sensing method according to one embodiment of the present invention.

FIG. 12 is a schematic diagram illustrating an example of a breath sensing system 100 according to one embodiment of the present invention.

FIG. 13 is a functional block diagram illustrating an example of the breath sensing system 100.

FIG. 14 is a diagram illustrating an example of a state of a main body 102.

FIG. 15 is a diagram illustrating another example of the state of the main body 102.

FIG. 16 is a diagram illustrating an example configuration of a state sensing unit 150.

FIG. 17 is a diagram illustrating an example of a display device 300 according to one embodiment of the present invention.

FIG. 18 is a diagram illustrating an example of a manipulator 310 of a display unit 210.

FIG. 19 is a block diagram illustrating an example of the display device 300 according to one embodiment of the present invention.

FIG. 20 is a diagram illustrating an example of a situation where the manipulator 310 is using an apparatus 400.

FIG. 21 is a diagram illustrating an example of a relationship between a CO₂ (carbon dioxide) concentration in a breath of the manipulator 310 and an expiratory volume of the manipulator 310.

FIG. 22 is a flowchart illustrating an example of a display method according to one embodiment of the present invention.

FIG. 23 is a diagram illustrating an example of a computer 2200 in which the breath sensing system 100 or the display device 300 according to one embodiment of the present invention may be entirely or partially embodied.

### DESCRIPTION OF EXEMPLARY EMBODIMENTS

Hereinafter, the present invention will be described through embodiments of the invention, but the following embodiments do not limit the invention according to the claims. In addition, not all of the combinations of features described in the embodiments are essential to the solution of the invention. In the present specification, the same parts in each figure are denoted by the same signs and numerals, and the descriptions thereof may be omitted. In addition, for convenience of description, some configurations may not be illustrated.

In the present specification, technical matters may be described using an orthogonal coordinate system of an X axis, a Y axis, and a Z axis. The orthogonal coordinate system merely specifies relative positions of components, and does not limit a particular direction. For example, the Z-axis direction is not limited to illustrating the height direction relative to the ground.

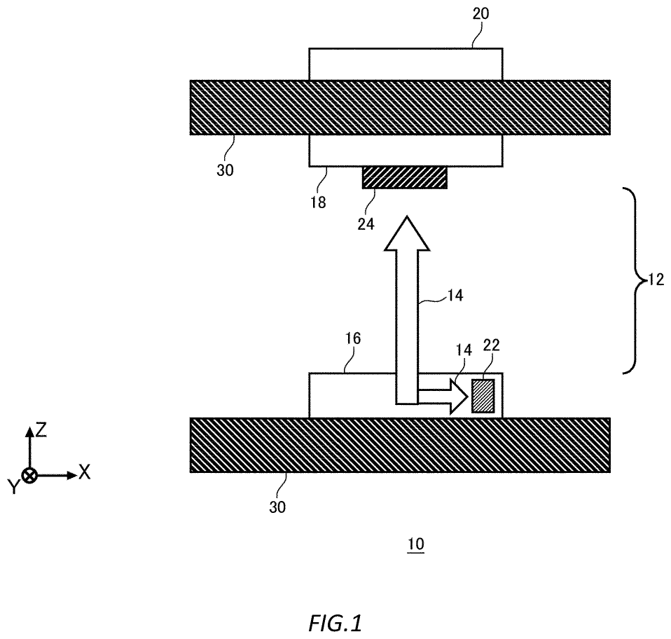

FIG. 1 is a diagram illustrating an example of a breath sensor 10 according to one embodiment of the present invention. The breath sensor 10 senses a breath generated by breathing. The breath sensor 10 includes a light emitting unit 16, a first light receiving unit 18, and an operating unit 20. The light emitting unit 16, the first light receiving unit 18, and the operating unit 20 may be attached to a support member 30. In the present example, the light emitting unit 16 is attached to one of support members 30 facing each other, and the first light receiving unit 18 and the operating unit 20 are attached to the other. In the present example, description is made by defining a surface that is parallel to a main surface of the support member 30 as the XY plane, and defining a direction that is orthogonal to the main surface of the support member 30 as the Z-axis direction.

The breath sensor 10 is an NDIR (non-dispersive infrared) sensor utilizing a specific absorption wavelength of carbon dioxide included in the breath. The light emitting unit 16 emits light 14 toward a path 12 through which the breath passes. As an example, the light emitting unit 16 is an IR-LED. The light 14 may be infrared light and may at least include an absorption wavelength of carbon dioxide. A part of the light 14 passes through the path 12 and enters the first light receiving unit 18. A part of the light 14 enters a second light receiving unit 22 described below.

The first light receiving unit 18 receives at least a part of the light 14 emitted by the light emitting unit 16, and outputs a first light reception signal according to a light reception result to the operating unit 20. The first light receiving unit 18 is a photodiode, as an example.

The operating unit 20 performs an operation on the first light reception signal. The operating unit 20 is an IC, as an example. Because the received amount of light in the absorption wavelength of carbon dioxide is decreased by carbon dioxide absorbing the light 14, the operating unit 20 can perform an operation to obtain a concentration of carbon dioxide or a value according to the concentration from the first light reception signal.

The breath sensor 10 may further include a thermometer. The operating unit 20 may receive temperature information of the breath sensor 10 from the thermometer. The operating unit 20 can further accurately perform an operation to obtain the concentration of carbon dioxide or the value according to the concentration from the first light reception signal by correcting the first light reception signal based on the temperature information.

The breath sensor 10 may include a second light receiving unit 22. Configurations of the light emitting unit 16 and the second light receiving unit 22 will be described below. The second light receiving unit 22 receives light 14, included in the light 14 emitted by the light emitting unit 16, which did not pass through the path 12 through which the breath passes, and outputs a second light reception signal according to the light reception result. The second light reception signal may be output to the operating unit 20. The operating unit 20 may correct the first light reception signal by using a signal value of the second light reception signal. The operating unit 20 may generate, as a detection signal, a signal obtained by dividing the signal value of the first light reception signal by the signal value of the second light reception signal. Output variation caused by a change in an amount of light from the light emitting unit 16 or a temperature can be corrected by providing the second light receiving unit 22.

The breath sensor 10 may further include an optical filter 24 located on a light path of the light 14 between the light emitting unit 16 and the first light receiving unit 18. The optical filter 24 in the present example is provided on a surface of the first light receiving unit 18. The optical filter 24 restricts the wavelength of light 14 that enters the first light receiving unit 18. The optical filter 24 is a band-pass filter, as an example.

Generally, the responsivity of the first light receiving unit 18 is changed by being influenced by the temperature. In a responsivity curve where the horizontal axis represents the wavelength of light and the vertical axis represents the responsivity of the first light receiving unit 18, the influence of the temperature appears as a shift in a horizontal axis direction of the responsivity curve. On the other hand, the responsivity curve has a wavelength range in which most of the responsivity values are significantly flat. By causing only light with a wavelength in said wavelength range to enter the first light receiving unit 18 using the optical filter 24, the influence on the responsivity can be suppressed even when the temperature of the first light receiving unit 18 changes.

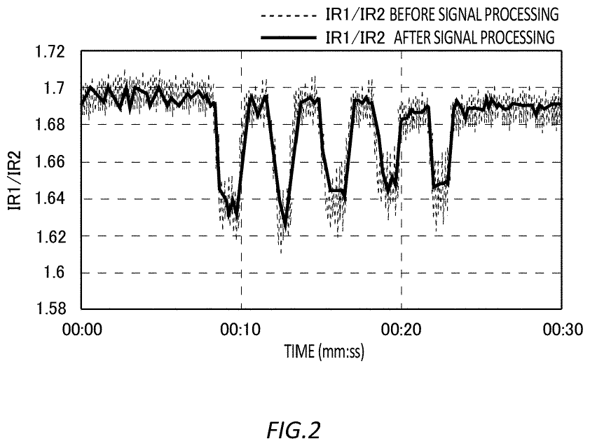

FIG. 2 is a diagram illustrating an example of a signal processing in the operating unit 20. FIG. 2 and subsequent figures indicate the first light reception signal by IR1 and the second light reception signal by IR2. In FIG. 2, the horizontal axis represents the time, and the vertical axis represents a value of a ratio between intensities of the first light reception signal and the second light reception signal. In the present specification, the ratio between the intensities of the first light reception signal and the second light reception signal may be referred to as a light reception signal ratio. The intensity of the first light reception signal may be simply referred to as a first light reception signal and the intensity of the second light reception signal may be simply referred to as a second light reception signal. As described above, by dividing the first light reception signal by the second light reception signal, the output variation caused by a change in the amount of light from the light emitting unit 16 and a temperature can be corrected.

Here, the first light reception signal and the second light reception signal may be a value corrected by using the temperature information described above in the operating unit 20. Unless the first light receiving unit 18 and the second light receiving unit 22 have same characteristics, the output variation can be further accurately corrected by respectively correcting the light reception signals by using the temperature.

Although the embodiments are also described in subsequent figures by using the light reception signal ratio between the first light reception signal and the second light reception signal, it is not necessary to use the second light reception signal. Instead of the light reception signal ratio, signal values of the first light reception signal may be used. In this case, the term “light reception signal ratio” may be used interchangeably with the “first light reception signal”. In the present specification, even in a case of performing a processing on the light reception signal ratio, a similar processing may be performed on the first light reception signal, and even in a case of performing a processing on the first light reception signal, a similar processing may be performed on the light reception signal ratio.

The operating unit 20 calculates a ratio between the first light reception signal and the second light reception signal that are input, and performs a signal processing on the light reception signal ratio. FIG. 2 illustrates denoising. In this figure, a dotted line indicates a light reception signal ratio before denoising and a solid line indicates a light reception signal ratio after denoising. The operating unit 20 performs the denoising by using a low-pass filter as an example. Because the amount of light received in the first light receiving unit 18 decreases due to absorption of carbon dioxide, the light reception signal ratio decreases when the breath is sensed. Further, because the breath quickly spreads into the air, the light reception signal ratio immediately increases (recovers) after the decrease. In this figure, a fluctuation of the light reception signal ratio around 10 seconds to 20 seconds corresponds to breathing. One valley-like portion in the light reception signal ratio corresponds to one time of breathing. In the example of FIG. 2, five valley-like portions in the waveform of the light reception signal ratio indicate the light reception signal ratio in five times of breathing.

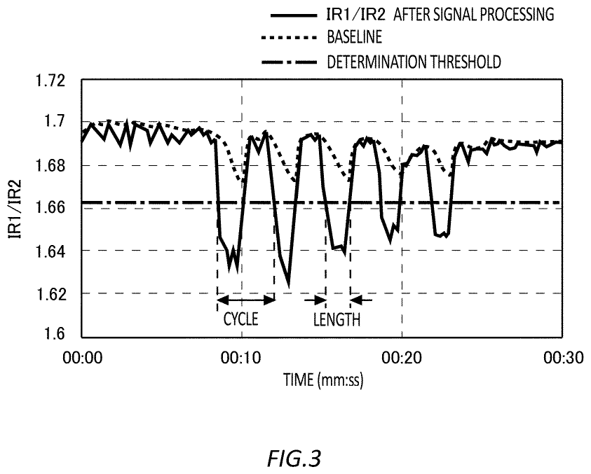

FIG. 3 is a diagram illustrating an example of a baseline calculation. Each axis in FIG. 3 is similar to that in FIG. 2. In this figure, a solid line illustrates a light reception signal ratio after a noise processing. The operating unit 20 calculates, from the light reception signal ratio, a baseline of a waveform of the first light reception signal. The dotted line in the figure indicates the calculated baseline.

The baseline refers to a signal waveform caused by a factor (disturbance) other than carbon dioxide included in the breath. The baseline is a waveform obtained by reducing or removing a component caused by the carbon dioxide concentration from the waveform shown by the solid line in FIG. 3. The baseline may include a component of the intensity of light 14 emitted by the light emitting unit 16 and a variation component caused by a factor other than a change in the concentration of carbon dioxide. When the breath sensor 10 senses a breath, the temperature of the breath may change the temperature of the light emitting unit 16 and the first light receiving unit 18, thereby changing their characteristics. The baseline may be a signal waveform that includes the influence of such change in the characteristics, but does not include the influence of carbon dioxide. While the baseline caused by disturbance the frequency of which out of the breathing cycle may include influences from humidity, degradation of elements, or the like, the baseline in the present specification is considered to include variation mainly caused by the temperature of the breath.

The operating unit 20 may calculate the baseline based on the light reception signal ratio. This allows the influence of the temperature of the breath to be appropriately reflected, because there is a correlation between the light reception signal ratio and the influence of the temperature by the breath. The baseline calculation value in FIG. 3 is varied in accordance with the breathing cycle because it is calculated from the light reception signal ratio, which represents the influence of the temperature caused by the breath. A parameter in the baseline calculation may be experimentally decided such that the change in the light reception signal ratio caused by a factor other than carbon dioxide is represented. The parameter is a coefficient b0 described below, as an example.

The operating unit 20 may calculate the baseline by removing the frequency component of the disturbance the frequency of which is out of the breathing cycle, for the temporal waveform of the light reception signal ratio. As an example, the operating unit 20 performs the calculation of the baseline by filtering represented by infinite impulse response (IIR). A process of an example of a baseline calculation in IIR filtering is shown in Table 1.

TABLE 1

| | |
|---|---|
| x0 | y0 = x0 |
| x1 | y1 = x1 × b0 + y0 × (1 − b0) |
| x2 | y2 = x2 × b0 + y1 × (1 − b0) |
| x3 | y3 = x3 × b0 + y2 × (1 − b0) |

x in the table represents signal values of the light reception signal ratio, and y represents the calculated value of the baseline. b0 is a filter coefficient. The numbers following x and y represent sampling points n. In Table 1, up to n=3 is shown.

The operating unit 20 calculates the value yn of the baseline for each signal value xn of the light reception signal ratio. As shown in Table 1, the calculated value yn of the baseline is calculated as a sum of a value obtained by multiplying a corresponding signal value xn by the coefficient b0 and a value obtained by multiplying the calculated value yn−1 of the immediately preceding baseline by (1−b0). That is, the coefficient b0 decides at what percentage the value of the corresponding signal value is to be reflected to the calculated value of a prior baseline. The coefficient b0 takes a value that is equal to or higher than 0, and equal to or lower than 1. By increasing the coefficient b0, the value of the corresponding signal value is more reflected, which makes it easier for the baseline to follow the variation in the signal values. Although, in the example of Table 1, a value yn−1 of one immediately preceding baseline is reflected for the calculated value yn of the baseline, in another example, values of a plurality of immediately preceding baselines may be reflected for the calculated value yn of the baseline.

The operating unit 20 may calculate the baseline by using a moving average (MA) filter as shown in Expression 1 below. In Expression 1, Yₙ represents the calculated value, Xₙ represents the signal values, M represents a number of sections, Fₛ represents the sampling frequency, and f_{c} represents the cut-off frequency. In the calculating method using the moving average, the operating unit 20 calculates an average value for each of certain fixed sections by moving from one section to another. The number of sections M is a number of the sections.

$$\begin{matrix}Expression 1 & \\ {Y}_{n} = \frac{1}{M} {\sum }_{i = 0}^{M - 1} {X}_{n - i} & ( 1 - 1 )\end{matrix}$$

$${f}_{c} = \frac{{F}_{s}}{2 M} \qquad ( 1 - 2 )$$

The operating unit 20 may calculate the baseline by using a weighted moving average (WMA) filter as shown in Expression 2 below. In Expression 2, Wᵢ represents a weight. M represents a number of sections as in Expression 1.

$$\begin{matrix}Expression 2 & \\ {Y}_{n} = \frac{{\sum }_{i = 0}^{M - 1} {w}_{i} {X}_{n - i}}{{\sum }_{i = 0}^{M - 1} {w}_{i}} & ( 2 )\end{matrix}$$

The operating unit 20 may calculate the baseline by using a finite impulse response (FIR) filter illustrated in Expression 3 below. In Expression 3, bi represents a filter coefficient. M represents a number of sections as in Expression 1.

$$\begin{matrix}Expression 3 & \\ {Y}_{n} = {\sum }_{i = 0}^{M - 1} {b}_{i} {X}_{n - i} & ( 3 )\end{matrix}$$

The operating unit 20 may calculate the baseline by using a Butterworth filter illustrated in Expression 4 below.

In Expression 4, N represents a filter order and s represents a complex frequency in the Laplace transform. Assuming that σ represents a real number, ω represents a frequency, and j represents an imaginary unit, s is represented as a sum of σ and jω.

$$\begin{matrix}Expression 4 & \\ H ( s ) = \frac{1}{\sqrt{1 + {( \frac{s}{{f}_{c}} )}^{2 N}}} & ( 4 )\end{matrix}$$

The operating unit 20 may calculate the baseline based on a signal value when signal variation in a predetermined period is small. For example, the signal variation is small in data at a time when no breathing takes place. For example, the predetermined period is a period in which five times of breathing in FIG. 2 takes place.

At least one of an upper limit value or a lower limit value may be set as the baseline. The baseline may be calculated by any of Table 1 and Expression 1 to Expression 4 depending on the signal value or the ambient temperature.

Here, an immediately preceding baseline calculation is not restricted to calculating the baseline of xn with xn−1. Any baseline calculation method may be used as long as it is difficult to follow the breathing signal. For example, for xn, baseline calculation may be performed by using all signals from xn−1 to further previous data xn-m, where m is a natural number. For example, if data has been acquired every 0.1 seconds, baseline calculation of the signal for xn may be performed by using data of signals xn−1, xn−2, xn−3, where m=3. The immediately preceding range may be decided such that it is difficult to follow the breathing signal, but can follow a slower temperature variation or the like. The calculation of the baseline may be performed based on the light reception signal ratio prior to Tn, or may be performed based on the light reception signal ratio at a particular period that is shorter than the breathing cycle prior to Tn.

The operating unit 20 removes the baseline from the light reception signal ratio. Removing the baseline may mean subtracting, from the signal value at each time, the value of the baseline at said time. By calculating and removing the baseline, the change in the signal values caused by factors other than carbon dioxide included in the breath can be corrected, allowing a more accurate measurement to be performed. In the present specification, the signal obtained after removing the baseline from the light reception signal ratio may be referred to as a determination signal.

The operating unit 20 senses a breath based on the determination signal. The operating unit 20 may determine that the breath is sensed when the value of the determination signal is lower than a determination threshold. The determination threshold may be preset. The operating unit 20 may calculate at least one of the cycle or length of the breathing. The operating unit 20 may calculate the cycle or length of the breathing in the period during which the value of the determination signal is lower than the determination threshold. The operating unit 20 may define, as the length of the breathing, one consecutive period during which the value of the determination signal is lower than the determination threshold. The operating unit 20 may calculate, as the cycle of the breathing, the sum of one consecutive period during which the value of the determination signal is higher than the determination threshold and one consecutive period during which the value of the determination signal is lower than the determination threshold. The operating unit 20 may calculate, as the cycle or length of the breathing, an average value within a predetermined measurement period. By calculating the cycle or length of the breathing, the emotion of the person breathing can be determined, such as whether the person is excited or relaxed. When measuring the cycle or length of the breathing, the amplitude of the determination signal does not need to be calculated and the measurement accuracy of the value of the determination signal does not need to be high, as long as the value of the determination signal and the determination threshold can be compared.

Here, setting the coefficient b0 in the infinite impulse response corresponds to removing a frequency component that is equal to or higher than a predetermined frequency from the signal value in the baseline calculation. Where said predetermined frequency is a first cut-off frequency, the relationship between the first cut-off frequency and the coefficient b0 is shown in Expression 5 below. In this expression, a1=b0−1.

$$\begin{matrix}Expression 5 & \\ {f}_{c} = \frac{1}{2 π} {cos}^{- 1} ( \frac{2 \times b {0}^{2} - 1 - a {1}^{2}}{2 \times a 1} ) & ( 5 )\end{matrix}$$

The operating unit 20 may calculate the baseline based on a frequency component of a temporal waveform in the light reception signal ratio which is lower than the first cut-off frequency that is set. The baseline can be calculated based on the frequency component of the light reception signal ratio which is lower than the first cut-off frequency by setting the coefficient b0 in the infinite impulse response. Specific examples of the coefficient b0 and the first cut-off frequency will be described below.

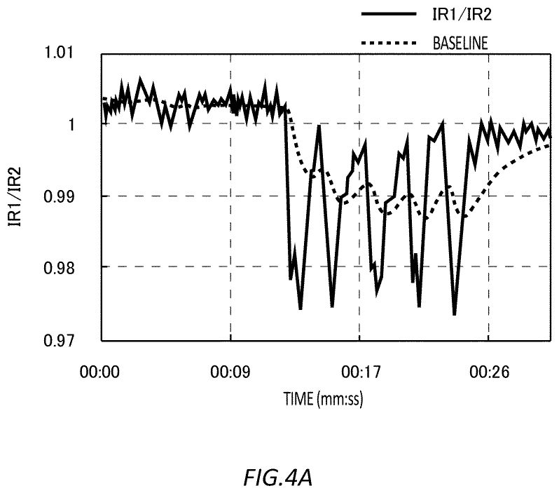

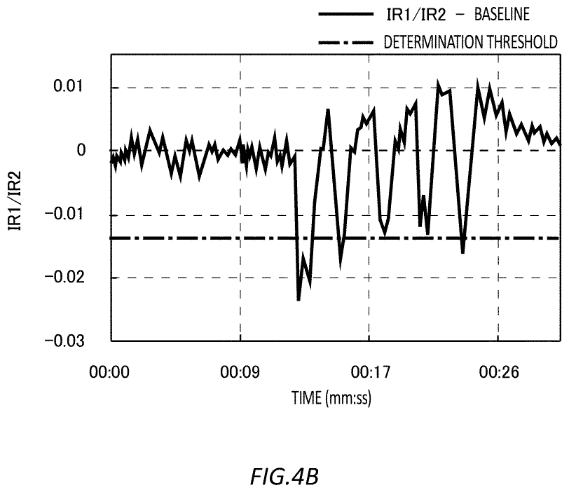

FIG. 4A is a diagram illustrating an example of a result of using a constant first cut-off frequency in the baseline calculation. The axes in FIG. 4A and solid lines and dotted lines in the figure are similar to those in FIG. 3. In the present example, the baseline calculation is performed with the first cut-off frequency being constant, for the value yn at each time in the baseline. For example, in the example of Table 1, b0 is a constant value for all n. In FIG. 4A, the followability of the baseline when the light reception signal ratio is increased is low. Thus, in a period where breathing is consecutive, the baseline does not recover to the original level (IR1/IR2=approximately 1 to 1.01), and remains at a relatively low level. In addition, immediately after the period in which breathing is consecutive ends, at some points, the baseline is lower than the light reception signal ratio.

FIG. 4B is a diagram illustrating an example of a determination signal obtained by calculating a difference between a light reception signal ratio and the baseline calculation value from FIG. 4A. Since the determination signal indicates a signal component caused by breath, the determination signal in a case where no breath exists ideally matches with a reference point (IR1/IR2=0). In addition, the determination signal in a case where breath exists ideally becomes smaller than the reference point. In FIG. 4B, a part of the determination signal is positioned above (on the positive side of) the reference point (IR1/IR2=0). In other words, the determination signal is shifted upward. Conceivably, this is because the followability of the baseline when the light reception signal ratio is increased is low, and the baseline is evaluated to be lower than the actual value.

One-dot chain lines in the figure indicate the determination threshold. As an example, it is determined that breath is sensed when the signal value is lower than the determination threshold. In FIG. 4B, only a part of the variation in the signal values caused by breath falls below the determination threshold, and a part of the breath may not be sensed. Therefore, it is preferable to more accurately calculate the breathing cycle or the like.

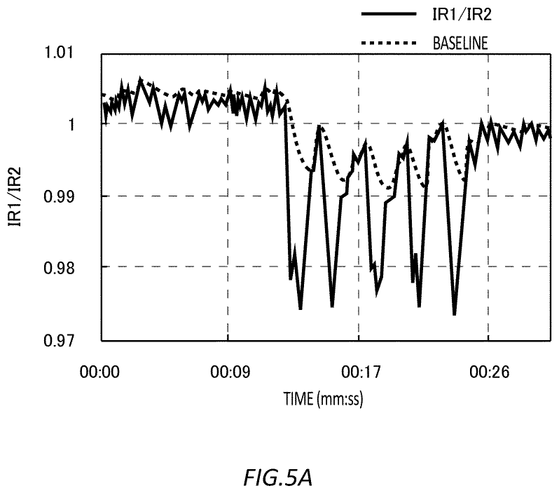

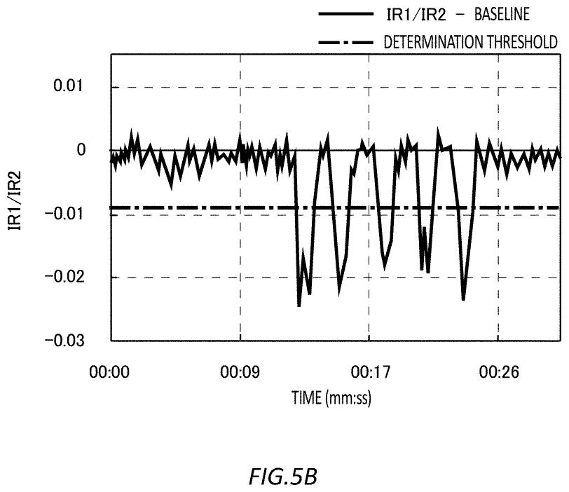

FIG. 5A is a diagram illustrating an example of a result of changing the first cut-off frequency in accordance with the magnitude relationship relative to a signal value at an immediately preceding timing in the baseline calculation. The first cut-off frequency may be set according to the change in the signal values of the light reception signal ratio. In the present example, the operating unit 20 changes the first cut-off frequency according to the change in the signal values of the light reception signal ratio. The operating unit 20 according to the present example sets the first cut-off frequency in a case where the signal value of the light reception signal ratio for which the value of the baseline is to be calculated is increased relative to the signal value at the immediately preceding timing in the temporal waveform of the light reception signal ratio to be higher compared to the first cut-off frequency in a case where it is decreased relative to the signal value at the immediately preceding timing.

Using the symbols in Table 1, the first cut-off frequency in a case where xn>xn−1 is set to be higher compared to the first cut-off frequency in a case where xn<xn−1. The operating unit 20 may set the coefficient b0 in a case where xn>xn−1 to be greater than the coefficient b0 in a case where xn<xn−1 to set the first cut-off frequency to be higher. By setting the first cut-off frequency to be higher, a relatively high frequency component in the signal waveform of the first light emitting signal can be reflected to the baseline. Thus, followability of the baseline when the signal value of the first light emitting signal is increased is improved. In FIG. 5A, the baseline closely follows the increase in the signal value of the first light emitting signal. Thus, the baseline returns near the original value (1 to 1.01), as compared to the example in FIG. 4A, in the period during which breathing is consecutive and the period immediately after.

When the breath flows into the breath sensor 10, the temperature of the breath may increase the temperature of an element after the carbon dioxide is sensed. Thus, it can be considered that an influence on the temperature characteristic of the element will appear in the temporal waveform of the baseline. Since the temperature of the element changes in synchronism with the breathing, the valley portion that is synchronous with the breathing as shown in FIG. 5A appears in the temporal waveform of the baseline. The influence of the temperature of the breath is considered to appear later than the sensing of carbon dioxide. By setting the first cut-off frequency as described above, in a period during which the signal value of the light reception signal ratio is reduced, the baseline calculation value is less likely to follow the variation in the signal values caused by sensing of carbon dioxide, which allows the baseline calculation value to be less influenced by carbon dioxide to appropriately reflect the influence of the temperature of the breath.

The operating unit 20 may set the first cut-off frequency in a case where the signal value of the light reception signal ratio for which the value of the baseline is to be calculated is increased relative to a signal value at a past timing in the temporal waveform of the light reception signal ratio to be higher compared to the first cut-off frequency in a case where it is decreased relative to the signal value at the past timing. The above-described signal value at the past timing may be an average value of the light reception signal ratio in a period that is shorter than the breathing cycle, which ends at the immediately preceding light reception signal ratio, may be the signal value of any light reception signal ratio in the period, or may be the signal value of all of the light reception signal ratios in the period. Using the symbols in Table 1, as an example, the first cut-off frequency in a case where xn is higher than the average value of xn−1, xn−2, and xn−3 may be set to be higher compared to the first cut-off frequency in a case where xn is lower than said average value.

The operating unit 20 may set the first cut-off frequency in a case where the signal value of the light reception signal ratio for which the value of the baseline is to be calculated is increased by 20% or more relative to a signal value at a past timing in the temporal waveform of the light reception signal ratio to be higher compared to the first cut-off frequency in a case where it is decreased by 20% or more relative to the signal value at the past timing.

FIG. 5B is a diagram illustrating an example of a determination signal calculated from a light reception signal ratio and the baseline illustrated in FIG. 5A. In FIG. 5B, other than minute noise components, the determination signals at all times are positioned below the reference point (IR1/IR2=0). Similarly to that in FIG. 4B, the one-dot chain line in the figure indicates the determination threshold. According to the present example, shifting of the determination signal can be suppressed, which allows breathing to be sensed and the cycle of length of the breathing to be calculated more accurately.

The operating unit 20 may set the first cut-off frequency in a case where the signal value of the light reception signal ratio for which the value of the baseline is to be calculated is increased relative to the signal value at the immediately preceding timing in the temporal waveform of the light reception signal ratio to be higher by ten times or more compared to the first cut-off frequency in a case where it is decreased relative to the signal value at the immediately preceding timing. As an example, the first cut-off frequency in a case where xn>xn−1 may be set to be equal to or higher than 1 Hz (for example, about 1.8 Hz (coefficient b0=0.24)), and the first cut-off frequency in a case where xn<xn−1 may be set to be equal to or lower than 0.1 Hz (for example, about 0.06 Hz (coefficient b0=0.01)). In this manner, the followability of the baseline is more improved. The above-described scale factor may be two times or more, may be five times or more, or may be 20 times or more. The first cut-off frequency may be equal to or lower than 3 Hz. Note that, the signal value at the above-described immediately preceding timing may be the signal value at the past timing described above. The operating unit 20 may set the first cut-off frequency in a case where the signal value of the light reception signal ratio for which the value of the baseline is to be calculated is increased by 20% or more relative to the signal value at the immediately preceding timing in the temporal waveform of the light reception signal ratio to be higher by ten times or more compared to the first cut-off frequency in a case where it is decreased by 20% or more relative to the signal value at the immediately preceding timing.

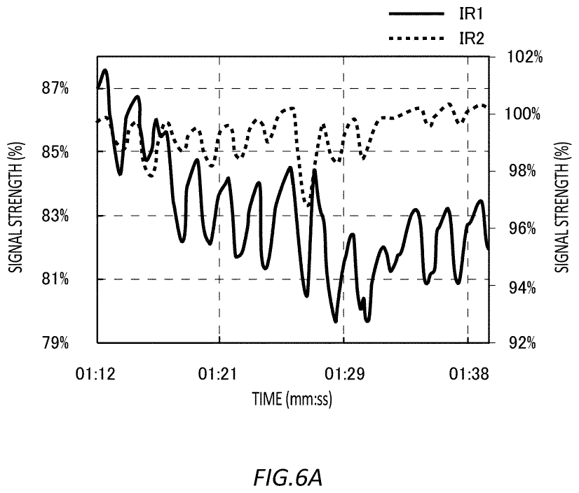

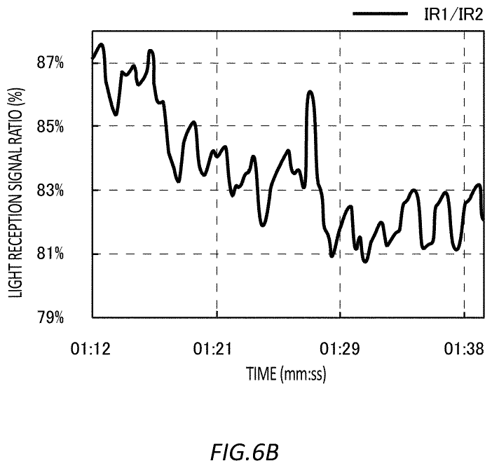

FIG. 6A is a diagram illustrating an example of the signal processing using equal cut-off frequencies respectively for the first light reception signal and a second light reception signal. In FIG. 6A, the horizontal axis represents time, the vertical axis on the left-hand side represents a signal strength (%) of the first light reception signal or IR1, and the vertical axis on the right-hand side represents a signal strength (%) of the second light reception signal or IR2. The first light reception signal is indicated by a solid line and the second light reception signal is indicated by a dotted line. A reference for the signal strength may be a signal value when no breath exists. The signal strength in the present example is a value in which a value of each signal is represented by percentage, where the value of the each signal when no breath exists is normalized as 100% (reference value). The difference between the reference value and the signal strength is the variation rate. In the case according to the present example, the following can be represented: the variation rate (%)=reference value (100%)−signal strength (%). The farther the signal strength becomes from 100%, the variation rate is increased and the variation from the reference value is larger. Note that, the reference value may be a value other than 100%.

The signal processing for removing a frequency component that is equal to or higher than a predetermined frequency from the signal value is performed on the first light reception signal and the second light reception signal in FIG. 6A. The predetermined frequency of the second light reception signal is used as a second cut-off frequency and the predetermined frequency of the first light reception signal is used as a third cut-off frequency. In the present example, the second cut-off frequency is equal to the third cut-off frequency.

The frequency of a typical breathing is about 0.25 Hz in the normal condition and about 0.42 Hz in the polypnea condition. The value of the third cut-off frequency is set to be higher than 0.42 Hz, and a fluctuation is seen in the first light reception signal by breathing. On the other hand, the second light reception signal fluctuates in a cycle close to the first light reception signal. Because the second light receiving unit 22 that outputs the second light reception signal receives the light 14 that did not pass the path 12 through which the breath passes, this can be considered to be an influence of the temperature of the breath. In particular, this tendency is intensified when the ambient temperature is low and a temperature difference between the ambient temperature and the breath is large.

FIG. 6B is a diagram illustrating an example of the light reception signal ratio calculated from the first light reception signal and the second light reception signal illustrated in FIG. 6A. In FIG. 6B, the horizontal axis represents the time and the vertical axis represents a light reception signal ratio (%). The light reception signal ratio has a waveform with a less cyclic property compared to the first light reception signal in FIG. 6A. In other words, the fluctuation of the first light reception signal is canceled out by the fluctuation of the second light reception signal. As a result, accuracy of breathing sensing deteriorates.

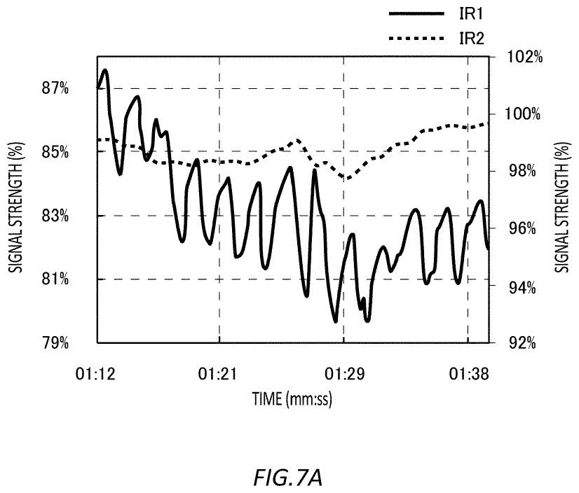

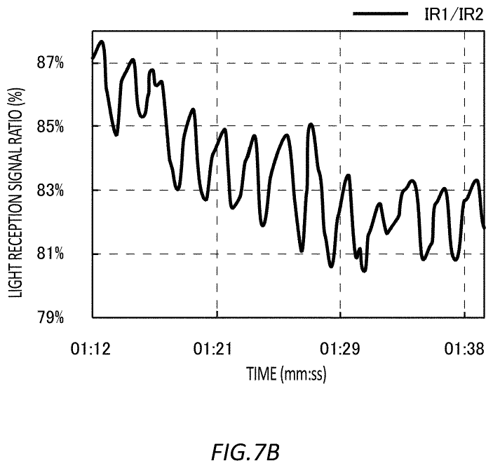

FIG. 7A is a diagram illustrating an example of a signal processing using different cut-off frequencies respectively for the first light reception signal and the second light reception signal. The second cut-off frequency and the third cut-off frequency in FIG. 7A differ from those in FIG. 6A. Other portions are similar to those in FIG. 6A. FIG. 7B is a diagram illustrating an example of the light reception signal ratio in FIG. 7A. FIG. 7B is similar to FIG. 6B except that the signal value in FIG. 7A is used in FIG. 7B.

The operating unit 20 generates a first light reception signal that is corrected, by using a frequency component of the second light reception signal lower than the second cut-off frequency that is set and a frequency component of the first light reception signal lower than the third cut-off frequency that is set. The first light reception signal that is corrected in the present example is a light reception signal ratio.

The second cut-off frequency in the present example is lower than the third cut-off frequency. Therefore, a cycle of the second light reception signal that is close to a cycle of the first light reception signal is removed. As a result, a light reception signal ratio is obtained to which the cycle of breathing in FIG. 7B is reflected. The second cut-off frequency may be lower than 0.25 Hz which is a frequency of breathing in a normal condition. The third cut-off frequency may be higher than 0.42 Hz which is a frequency in a polypnea condition. The operating unit 20 may perform a baseline calculation as described above based on the light reception signal ratio.

The operating unit 20 may generate the first light reception signal that is corrected by changing the filter order in Expression 4 instead of the cut-off frequency. When denoising, the operating unit 20 may change a type of the low-pass filter. The operating unit 20 may change an attenuation slope of the low-pass filter. The attenuation slope is an index indicating a steepness of signal attenuation from a pass band to a stop band in a transition portion of a filter.

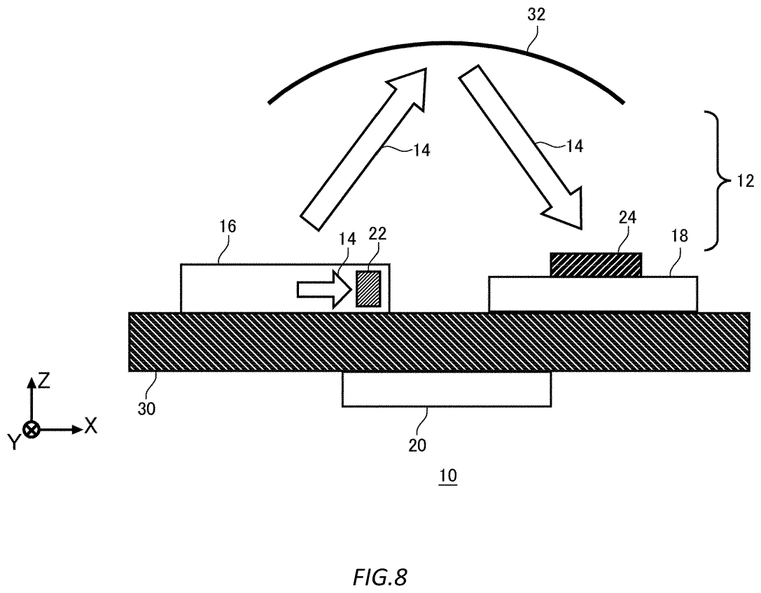

FIG. 8 is a diagram illustrating another example of the breath sensor 10 according to one embodiment of the present invention. In the breath sensor 10 according to the present example, the light emitting unit 16, the first light receiving unit 18, and the operating unit 20 are attached to a same support member 30. The breath sensor 10 according to the present example includes a mirror 32. The path 12 through which the breath passes extends between the support member 30 and the mirror 32. In the breath sensor 10 according to the present example, a part of the light 14 emitted by the light emitting unit 16 is reflected by the mirror 32 and the reflected light 14 is received by the first light receiving unit 18. Other portions may be similar to those of the breath sensor 10 in FIG. 1. Such a configuration can make the breath sensor 10 smaller.

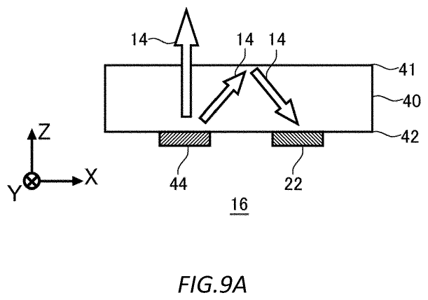

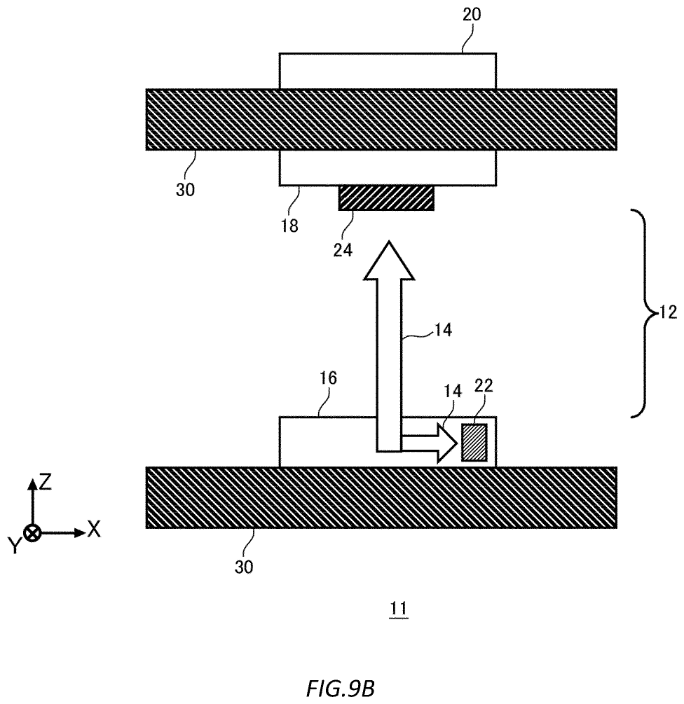

FIG. 9A is a diagram illustrating an example configuration of the light emitting unit 16. The light emitting unit 16 according to the present example includes a substrate 40, a light emitting element 44, and a second light receiving unit 22. The substrate 40 may be transparent to the light 14. When a breath is to be sensed, the substrate 40 is a GaAs substrate, as an example.

The substrate 40 includes a first main surface 41 and a second main surface 42. The first main surface 41 and the second main surface 42 are two main surfaces that constitute the substrate 40. In the present example, of the two main surfaces, the first main surface 41 is the main surface on the positive side in the Z-axis direction, and the second main surface 42 is the main surface on the negative side in the Z-axis direction. The first main surface 41 faces the path 12 through which the breath passes.

The light emitting element 44 and the second light receiving unit 22 may be provided on the same substrate. The light emitting element 44 and the second light receiving unit 22 of the present example are provided on the second main surface 42 of the substrate 40. The light emitting element 44 includes a laminated structure unit of PN junction or PIN junction. By supplying electrical power to this laminated structure unit, the light emitting element 44 operates as LED, and emits the light 14 with a wavelength according to the band gap of the material of the laminated structure unit. When breath is to be sensed, the light emitting element 44 may use, as the laminated structure unit, InAlSb that can perform an output near an absorption wavelength of carbon dioxide. The light emitting element 44 described above is referred to as a quantum infrared light emitting element.

A part of the light 14 proceeds through the substrate 40, passes through the first main surface 41, and is released to the path 12. On the other hand, other parts of the light 14 proceed through the substrate 40, are reflected on the first main surface 41, and enter the second light receiving unit 22.

The second light receiving unit 22 receives the light 14 that proceeded through the substrate 40. The second light receiving unit 22 receives the light 14 that did not pass through the path 12 through which the breath passes. In this manner, the output variation caused by the change in the amount of light from the light emitting element 44 and temperature can be corrected.

The second light receiving unit 22 may also include a laminated structure unit. The laminated structure unit of the second light receiving unit 22 may be a diode structure of PN junction or PIN junction. The laminated structure unit and the material of the second light receiving unit 22 may be similar to the laminated structure unit and the material of the light emitting element 44. In this manner, the temperature characteristic of the second light receiving unit 22 and the temperature characteristic of the light emitting element 44 can be matched with each other. The second light receiving unit 22 described above is referred to as a quantum infrared light receiving element.

FIG. 9B is a diagram illustrating an example of a gas sensor 11 according to one embodiment of the present invention. While the embodiments of the breath sensor 10 have been described with reference to FIG. 1 to FIG. 9A, the above-described embodiments are not limited to a sensor which senses a breath. The above-described embodiment can be widely applied to a gas sensor 11 which senses a gas to be sensed.

The gas sensor 11 may have a configuration as illustrated in FIG. 8 or FIG. 9A. The breath sensor 10 described with reference to FIG. 2 to FIG. 9A may function as the gas sensor 11. For example, the gas sensor 11 may include a light emitting unit 16, a first light receiving unit 18, and an operating unit 20. The operating unit 20 of the gas sensor 11 may perform the signal processing described with reference to FIG. 2, may perform the baseline calculation and removal described with reference to FIG. 3 to FIG. 5B, may perform the signal processing described with reference to FIG. 6A to FIG. 7B, or may perform a combination thereof.

The operating unit 20 of the gas sensor 11 may calculate a baseline of the waveform of the first light reception signal from the light reception signal ratio based on the frequency component of the light reception signal ratio lower than the first cut-off frequency that is set, and remove the baseline from the light reception signal ratio. In the baseline calculation, the operating unit 20 of the gas sensor 11 may change the first cut-off frequency in accordance with a change in the signal values of the light reception signal ratio.

If the above-described embodiments are applied, the influence of the disturbance other than the target to be sensed can be removed, in particular when the output value of the gas sensor 11 tends to change over time and the orientation of the output variation when sensing the target to be sensed is fixed. As an example, it is effective for a methane gas sensor or the like installed at an outdoor location where the ambient temperature tends to vary.

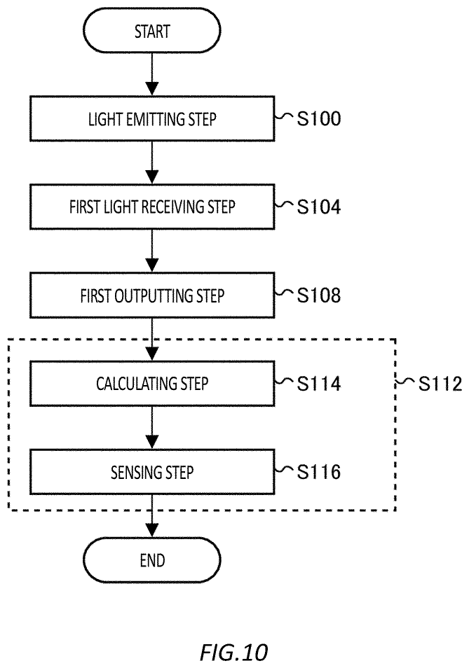

FIG. 10 is a flowchart illustrating an example of a breath sensing method according to one embodiment of the present invention. The breath sensing method according to one embodiment of the present invention will be described by using the breath sensor 10 in FIG. 1 as an example. The breath sensing method includes a light emitting step S100, a first light receiving step S104, a first outputting step S108, and an operating step 112. The operating step 112 includes a calculating step S114 and a sensing step S116.

In the light emitting step S100, the light emitting unit 16 emits the light 14 toward the path 12 through which the breath passes. In the first light receiving step S104, the first light receiving unit 18 receives at least a part of the light 14 emitted in the light emitting step S100. In the first outputting step S108, the first light receiving unit 18 outputs a first light reception signal according to the light reception result in the first light receiving step S104. In the operating step 112, the operating unit 20 performs an operation on the first light reception signal output in the first outputting step S108.

In the calculating step S114, the operating unit 20 calculates a baseline of the waveform of the first light reception signal based on the frequency component of the first light reception signal lower than the first cut-off frequency that is set. In the sensing step S116, the operating unit 20 senses a breath based on the signal obtained by removing the baseline calculated in the calculating step S114 from the first light reception signal.

In the operating step 112, the operating unit 20 may calculate at least one of a cycle or a length of breathing. The first cut-off frequency may be set according to the change in the signal values of the first light reception signal.

In the operating step 112, the operating unit 20 may set the first cut-off frequency in a case where the signal value of the first light reception signal for which the value of the baseline is to be calculated is increased relative to a signal value at a past timing in the temporal waveform of the first light reception signal to be higher compared to the first cut-off frequency in a case where it is decreased relative to the signal value at the past timing. In the operating step 112, the operating unit 20 may set the first cut-off frequency in a case where the signal value of the first light reception signal for which the value of the baseline is to be calculated is increased by 20% or more relative to the signal value at the past timing in the temporal waveform of the first light reception signal to be higher compared to the first cut-off frequency in a case where it is decreased by 20% or more relative to the signal value at the past timing.

In the operating step 112, the operating unit 20 may set the first cut-off frequency in a case where the signal value of the first light reception signal for which the value of the baseline is to be calculated is increased relative to the signal value at the immediately preceding timing in the temporal waveform of the first light reception signal to be higher by ten times or more compared to the first cut-off frequency in a case where it is decreased relative to the signal value at the immediately preceding timing. In the operating step 112, the operating unit 20 may set the first cut-off frequency in a case where the signal value of the first light reception signal for which the value of the baseline is to be calculated is increased by 20% or more relative to the signal value at the immediately preceding timing in the temporal waveform of the first light reception signal to be higher by ten times or more compared to the first cut-off frequency in a case where it is decreased by 20% or more relative to the signal value at the immediately preceding timing.

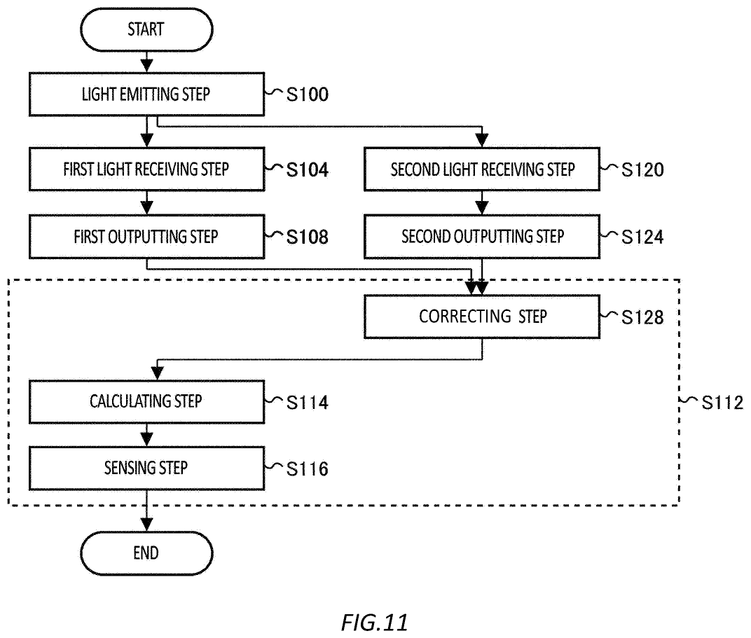

FIG. 11 is a flowchart illustrating another example of the breath sensing method according to one embodiment of the present invention. The breath sensing method in FIG. 11 is different from the breath sensing method in FIG. 10 in that it further includes a second light receiving step S120, a second outputting step S124, and a correcting step S128. The operating step 112 may include the correcting step S128.

In the second light receiving step S120, the second light receiving unit 22 receives the light 14, included in the light 14 emitted by the light emitting unit 16, which did not pass the path 12 through which the breath passes. In the second outputting step S124, the second light receiving unit 22 outputs a second light reception signal according to a light reception result in the second light receiving step S120. In the correcting step S128, the operating unit 20 corrects the first light reception signal based on the signal value of the second light reception signal. In calculating step S114 according to the present example, the operating unit 20 calculates a baseline of a waveform of the first light reception signal based on a frequency component, which is lower than the first cut-off frequency that is set, of the first light reception signal corrected in the correcting step S128.

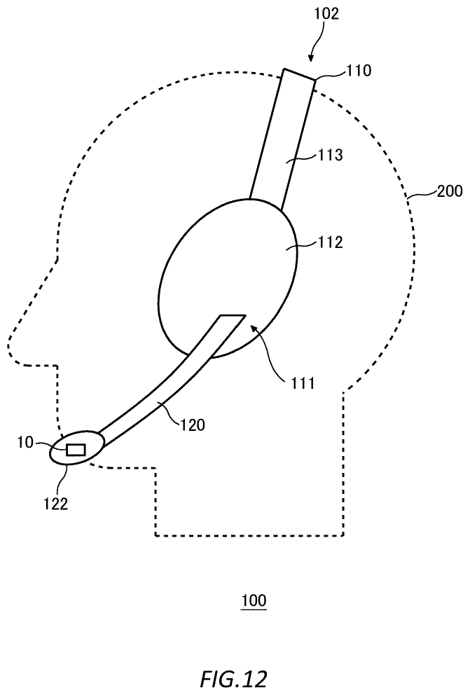

FIG. 12 is a schematic diagram illustrating an example of a breath sensing system 100 according to one embodiment of the present invention. The breath sensing system 100 senses a breath of a user 200. The breath sensing system 100 may be a part or an entirety of a device worn by the user 200. The breath sensing system 100 according to the present example is an entirety of a headset to be worn on a head of the user 200.

The breath sensing system 100 main body 102 illustrated in FIG. 12 includes the breath sensor 10. The breath sensor 10 is provided in the main body 102 and senses the breath of the user 200. The breath is air exhaled from the user 200 by breathing. The breath sensor 10 may sense the breath of the user 200 based on a concentration of carbon dioxide. The breath sensor 10 may measure the concentration of carbon dioxide in a measurement space by measuring an absorption intensity of infrared rays that pass the measurement space.

The breath sensor 10 may determine that the breath of the user 200 is sensed, when the concentration of carbon dioxide measured is equal to or higher than a reference value. The breath sensor 10 may sense a cycle of breathing or a length of breathing of the user 200 based on the result of sensing the breath. The breath sensor 10 may calculate a period during which the concentration of carbon dioxide is continuously equal to or higher than the reference value as a length of one time of breathing. The breath sensor 10 may calculate a length as a cycle of breathing by dividing a unit of time by a number of times of breathing that took place within the unit of time.

The main body 102 according to the present example includes a fixed part 110 and a movable part 120. As an example, the fixed part 110 is worn on the head of the user 200. The fixed part 110 may include a sound generating unit 112 which generates a sound. The sound generating unit 112 may include pad portions to be in contact with ears of the user 200 when wearing the main body 102 and a speaker provided in the pad portions. The fixed part 110 may include two sound generating units 112 corresponding to both ears of the user 200 and an arm part 113 which connects the two sound generating units 112.

The movable part 120 is provided to be movable relative to the fixed part 110. In other words, a position of at least a portion of the movable part 120 can be changed relative to at least a portion of the fixed part 110. The movable part 120 according to the present example includes a bar-shaped portion extending from the fixed part 110. The bar-shaped portion is connected to a connector 111 in the fixed part 110. The bar-shaped portion can be rotated relative to the fixed part 110 about a connector 111 as a rotational axis.

The breath sensor 10 may be provided in the movable part 120. A position of the breath sensor 10 relative to the fixed part 110 may be changeable in accordance with a movement of the movable part 120. The breath sensor 10 according to the present example is provided on an end of the movable part 120 opposite to the connector 111. The sound sensing unit 122 which senses a sound is provided on the end. For example, the sound sensing unit 122 is a microphone. The breath sensor 10 may be provided in the sound sensing unit 122. By locating the sound sensing unit 122 and the breath sensor 10 to be close to each other, the breath of the user 200 may be easily sensed because the breath sensor 10 tends to be located close to a mouth of the user 200.

The main body 102 may further include a display unit which displays information. For example, the display unit may be a head-mounted display connected to the fixed part 110. The display unit may be provided to be movable relative to the fixed part 110. The breath sensor 10 may be provided in the display unit.

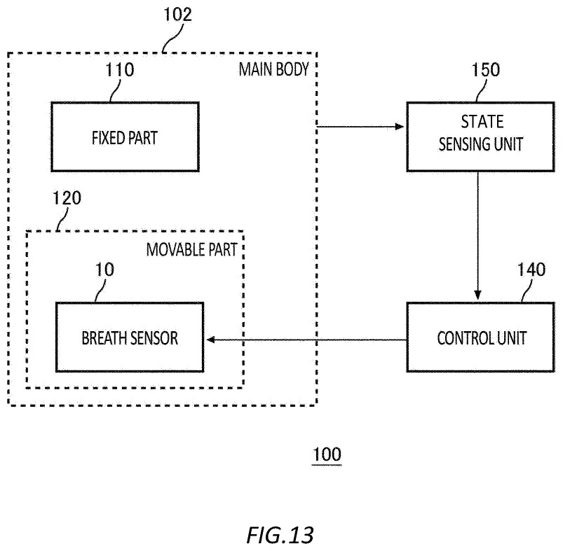

FIG. 13 is a functional block diagram illustrating an example of the breath sensing system 100. The breath sensing system 100 according to the present example includes a main body 102, a state sensing unit 150, and a control unit 140. The main body 102 is similar to the example described in FIG. 12. The main body 102 according to the present example includes a fixed part 110 and a movable part 120. The breath sensor 10 is provided in the movable part 120. The operating unit 20 as shown in FIG. 1 may function as the control unit 140. The operating unit 20 may function as a part of the state sensing unit 150.

While the state sensing unit 150 and the control unit 140 are located exterior to the main body 102 in FIG. 13, the state sensing unit 150 and the control unit 140 may be provided in the main body 102. The control unit 140 may be provided on a same substrate as the breath sensor 10. The control unit 140 may receive a signal from the state sensing unit 150. The control unit 140 may receive the signal by wire or wirelessly by a signal transmitting means.

The state sensing unit 150 senses a state of at least one of the main body 102 or the breath sensor 10. The state of at least one of the main body 102 or the breath sensor 10 may be simply referred to as a state of the main body 102 in the present specification. The state of the main body 102 may include at least one of a positional state indicating a position of a part, such as the movable part 120 or the like, relative to the fixed part 110, an operating state indicating an electrical state of the main body 102, or an environmental state indicating the surrounding environment of the main body 102.

The positional state may be information indicating a state of a part which affects the position of the breath sensor 10 relative to the fixed part 110. The positional state may be information directly indicating the relative position of the breath sensor 10.

The operating state may be information indicating presence or absence, a magnitude, a frequency, or the like of source power, a control signal, or the like input to the main body 102. The operating state may be information indicating presence or absence, a magnitude, a frequency, or the like of an electrical signal generated by each part of the main body 102. The operating state may be information indicating the magnitude of power consumption by the main body 102.

More specifically, the operating state may be information indicating whether a control signal for causing the sound generating unit 112 to generate a sound is input to the main body 102 or may be information indicating whether a control signal for causing the display unit to display information is input to the main body 102. The sound sensing unit 122 converts the sensed sound into an electrical signal. The operating state may be information indicating presence or absence, a magnitude, a frequency, or the like of an electrical signal output from the sound sensing unit 122. When the source power of the main body 102 is supplied from the storage battery of the main body 102, the operating state may be information indicating a remaining capacity of the storage battery.

The environmental state may be information indicating at least one of a temperature, humidity, air pressure, wind speed, or the like surrounding the main body 102. The state sensing unit 150 may have a sensor which senses the information above.

The breath sensor 10 has a plurality of operating modes having different power consumption. The breath sensor 10 according to the present example has, as the operating modes, an active state in which the breath can be sensed and a standby state in which power consumption is lower than the active state. For example, a light emitting element of the breath sensor 10 continuously emits light such as infrared light in the active state. The power consumption per unit of time of the light emitting element in the standby state is smaller than the power consumption per unit of time of the light emitting element in the active state.

A light emission cycle of the light emitting element in the standby state may be longer than a light emission cycle of the light emitting element in the active state. A light emission strength of the light emitting element in the standby state may be lower than a light emission strength of the light emitting element in the active state. Light emission of the light emitting element may be stopped in the standby state.

The control unit 140 causes the breath sensor 10 to transition between operating modes based on a state of at least one of the main body 102 or the breath sensor 10 sensed by the state sensing unit 150. The control unit 140 may control the breath sensor 10 to transition between the operating modes so as to decrease the power consumption of the breath sensor 10 when the state of the main body 102 satisfies a preset condition compared to when the state of the main body 102 does not satisfy the condition. For example, the condition indicates that the breath sensor 10 is in a state where it should not sense the breath.

For example, when the breath sensor 10 is located at a position away from the mouth of the user 200, the breath sensor 10 cannot accurately sense the breath of the user 200. The control unit 140 may control the breath sensor 10 to transition between the operating modes so as to decrease the power consumption of the breath sensor 10 when a positional state of the main body 102 satisfies the preset condition. Thus, the power consumption of the breath sensor 10 can be reduced.

When application software for operating the main body 102 does not request sensing of the breath of the user 200, the breath sensor 10 may not sense the breath. The control unit 140 may control the breath sensor 10 to transition between the operating modes so as to decrease the power consumption of the breath sensor 10 when an operating state indicated by the control signal or the like to the main body 102 satisfies the preset condition. Thus, the power consumption of the breath sensor 10 can be reduced. The control unit 140 may control the breath sensor 10 to transition between the operating modes so as to decrease the power consumption of the breath sensor 10 when the remaining capacity of the storage battery of the main body 102 is equal to or lower than a preset reference value. Thus, consumption of the remaining capacity of the storage battery can be reduced.

When the user 200 is uttering, the breath sensor 10 cannot accurately sense information related to the breath of the user 200, for example, a breathing cycle or the like. The control unit 140 may control the breath sensor 10 to transition between the operating modes so as to decrease the power consumption of the breath sensor 10 when an amplitude or the like of the electrical signal output from the sound sensing unit 122 satisfies the preset condition. Thus, the power consumption of the breath sensor 10 can be reduced.

The breath sensor 10 may be unable to accurately sense the breath due to a temperature, humidity, air pressure, or wind speed surrounding the breath sensor 10. For example, an error may occur in the detection result of the breath sensor 10 due to a temperature characteristic of the breath sensor 10. An error may occur in the detection result of the breath sensor 10 due to dew condensation in an optical element such as the light emitting element and the light receiving element of the breath sensor 10. An error may occur in the detection result of the breath sensor 10 due to an excessively high wind speed which causes the breath to easily spread in a space in a course of traveling from the mouth of the user 200 to the breath sensor 10. The control unit 140 may control the breath sensor 10 to transition between the operating modes so as to decrease the power consumption of the breath sensor 10 when an environmental state of the main body 102 satisfies the preset condition. Thus, the power consumption of the breath sensor 10 can be reduced.

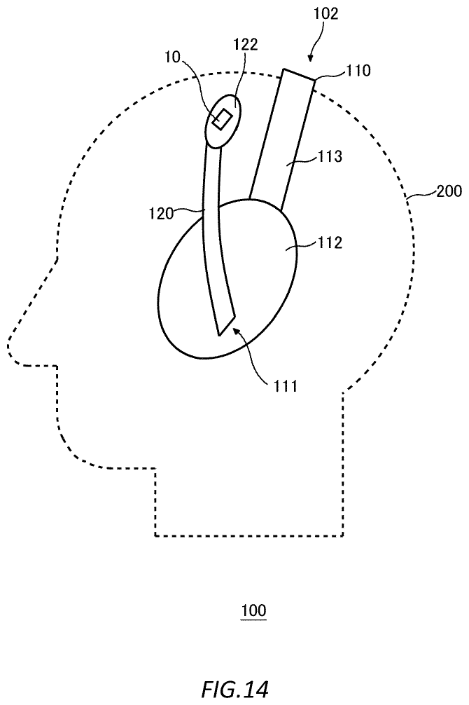

FIG. 14 is a diagram illustrating an example of the positional state of the main body 102. When compared to the example illustrated in FIG. 12, a rotational angle of the movable part 120 of the present example relative to the fixed part 110 is changed. Therefore, the breath sensor 10 according to the present example is located farther away from the mouth of the user 200 than the example illustrated in FIG. 12. In such a case, the breath sensor 10 cannot accurately sense the breath of the user 200.

The control unit 140 may control the operating modes of the breath sensor 10 according to whether a state of the movable part 120 satisfies a preset condition. The control unit 140 may control the breath sensor 10 to transition to the standby state when the rotational angle of the movable part 120 is out of a preset range.

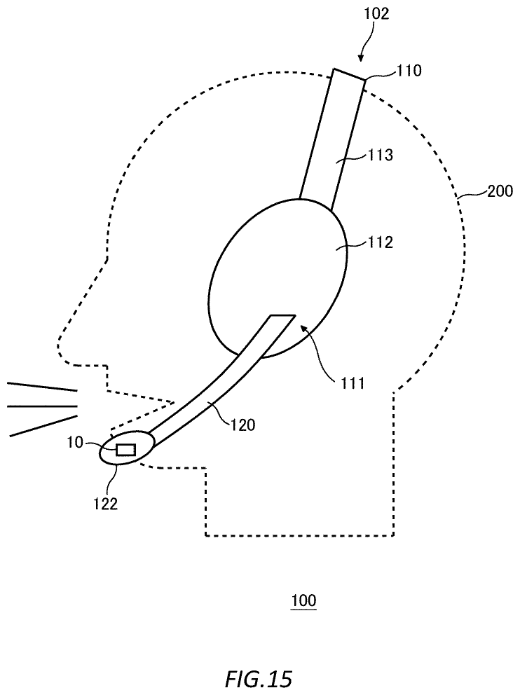

FIG. 15 is a diagram illustrating another example of the state of the main body 102. The user 200 is uttering in the present example. The breath sensed by the breath sensor 10 when the user 200 is uttering may be different from the breath when the user 200 is not uttering. Therefore, when it is about to sense a length, a cycle, or the like of breathing in a state where the user 200 is not uttering, the breath sensor 10 cannot accurately sense the length, the cycle, or the like of breathing, even though the breath sensor 10 is operating, in a state where the user 200 is uttering.

The control unit 140 may control the operating modes of the breath sensor 10 according to whether the electrical signal output from the sound sensing unit 122 satisfies a preset condition. The control unit 140 may control the breath sensor 10 to transition to the standby state when a magnitude of the amplitude of the electrical signal, which corresponds to a magnitude of an utterance of the user 200, is equal to or higher than a reference value.

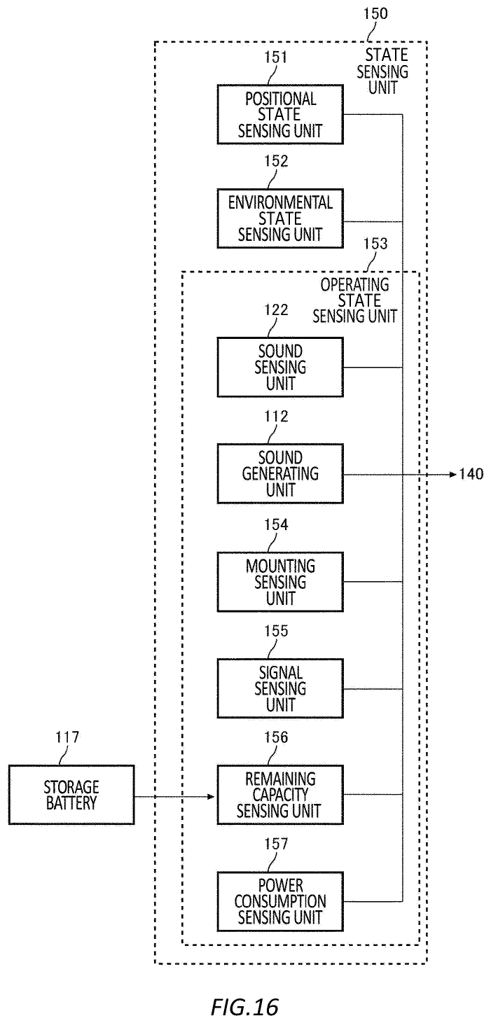

FIG. 16 is a diagram illustrating an example configuration of the state sensing unit 150. The main body 102 according to the present example includes a storage battery 117 which supplies source power to the main body 102. The state sensing unit 150 according to the present example includes at least one of a positional state sensing unit 151, an environmental state sensing unit 152, or an operating state sensing unit 153, or all of them.

The positional state sensing unit 151 senses the positional state described above. The positional state sensing unit 151 may include a sensor which senses the positional state such as the rotational angle of the movable part 120 or the like.

The environmental state sensing unit 152 senses the environmental state described above. The environmental state sensing unit 152 may include at least one of a temperature sensor, a humidity sensor, a pressure sensor, or a wind speed sensor.

The operating state sensing unit 153 senses the operating state described above. The sound sensing unit 122 and the sound generating unit 112 illustrated in FIG. 12 or the like may be included in the operating state sensing unit 153. The operating state sensing unit 153 according to the present example includes at least one of a sound sensing unit 122, a sound generating unit 112, a mounting sensing unit 154, a signal sensing unit 155, a remaining capacity sensing unit 156, or a power consumption sensing unit 157, or all of them.

The sound sensing unit 122 senses a sound. The control unit 140 may control the operating modes of the breath sensor 10 based on a state of the sound sensed by the sound sensing unit 122. The control unit 140 may control the breath sensor 10 to transition to the standby state when a magnitude of the sound sensed by the sound sensing unit 122 is equal to or higher than the set reference value. The control unit 140 may control the breath sensor 10 to transition to the active state when the magnitude of the sound sensed by the sound sensing unit 122 is lower than the set reference value.

The control unit 140 may control the breath sensor 10 to transition to the standby state when the sound sensed by the sound sensing unit 122 is a sound from the user 200. The control unit 140 may control the breath sensor 10 to transition to the active state when the sound from the user 200 is sensed no more. The control unit 140 may determine whether the sound from the user 200 is sensed based on a magnitude of a preset frequency component included in the electrical signal output from the sound sensing unit 122. For example, the frequency component may be 80 Hz or more and 1 kHz or less.

The sound generating unit 112 generates a sound. The control unit 140 may control the operating modes of the breath sensor 10 based on a state of the sound being generated or about to be generated by the sound generating unit 112. For example, if it is desired to sense the breath of the user 200 when listening to a sound such as music, the breath sensor 10 may be controlled to transition from the standby state to the active state when the sound generating unit 112 is generating or about to generate the sound such as music. In another example, if it is desired to sense the breath of the user 200 in a quiet state, the breath sensor 10 may be controlled to transition from the standby state to the active state when the magnitude of the sound which the sound generating unit 112 is generating or about to generate is equal to or lower than the reference value.

The mounting sensing unit 154 determines whether the main body 102 is worn by the user 200. The mounting sensing unit 154 may be a distance sensing unit which senses a distance between the main body 102 and another object, for example, the user 200. The mounting sensing unit 154 may determine that the main body 102 is worn when the distance is equal to or less than a reference value and determine that the main body 102 is not worn when the distance is larger than the reference value. The control unit 140 may control the breath sensor 10 to transition to the standby state when the main body 102 is not worn. The control unit 140 may control the breath sensor 10 to transition to the active state when the main body 102 is worn.

The signal sensing unit 155 senses a state of an input signal input to the main body 102 or an output signal generated by the main body 102. The input signal may be a signal for designating the state of the breath sensor 10. The control unit 140 may control the breath sensor 10 to transition to the active state or to the standby state based on the input signal. The input signal may be a signal for controlling the sound generating unit 112 or the display unit.

The main body 102 may change its state according to the control signal from the application software running on a computer situated inside or outside of the main body 102. The control signal may control the sound generating unit 112 or the display unit. The signal sensing unit 155 may control the operating modes of the breath sensor 10 based on the control signal.

The control unit 140 may control the breath sensor 10 to transition to the active state or to the standby state when a control signal for setting the sound generating unit 112 or the display unit to a predetermined state is input. The predetermined state may be preset in the control unit 140. The control unit 140 may control the breath sensor 10 to transition to the active state when a maximum sound volume generated by the sound generating unit 112 or a maximum luminance of an image displayed on the display unit is higher than a predetermined reference value. In another example, the control unit 140 may control the breath sensor 10 to transition to the standby state when the maximum sound volume generated by the sound generating unit 112 or the maximum luminance of the image displayed on the display unit is larger than the predetermined reference value. The application software may designate a state to which the breath sensor 10 is controlled to transition, according to a result of comparison between the state value and the reference value.

The control unit 140 may control the operating modes of the breath sensor 10 based on a type of the application software described above. The control signal may include information indicating the type of the application software. For example, when the application software is a game application, the control unit 140 controls the breath sensor 10 to transition to the active state. When the application software is a conference application for holding a conference with users on the web, the control unit 140 may control the breath sensor 10 to transition to the standby state.

The remaining capacity sensing unit 156 may sense the remaining capacity of electrical power that can be output from the storage battery 117, and may sense a state of charging of the storage battery 117, for example, a start of charging, a stop of charging, or a middle of charging, and may sense both of the remaining capacity and the state of charging. The control unit 140 may control the breath sensor 10 based on the remaining capacity of the storage battery 117 and may control the breath sensor 10 based on the state of charging of the storage battery 117. The control unit 140 may control the breath sensor 10 to transition to the standby state when the remaining capacity is lower than the reference value. The control unit 140 may control the breath sensor 10 to transition to the active state when the start of charging of the storage battery 117 is sensed. The control unit 140 may control the breath sensor 10 to transition to the standby state for rapid charging of the storage battery 117 when the start of charging of the storage battery 117 is sensed.

The power consumption sensing unit 157 senses power consumption of the main body 102. The main body 102 may have a plurality of operating states having different power consumption. For example, the main body 102 has an activated state where the source power is input and an inactivated state where the source power is not input. The control unit 140 may control the operating modes of the breath sensor 10 based on the operating state of the main body 102. The control unit 140 may control the breath sensor 10 to the active state when the main body 102 is in the activated state and control the breath sensor 10 to the standby state when the main body 102 is in the inactivated state. The control unit 140 may transition the breath sensor 10 to the standby state when the power consumption of the main body 102 is equal to or less than the set reference value.

The operating state sensing unit 153 may sense the operating state of the main body 102 based on the breath sensed by the breath sensor 10. In other words, the breath sensor 10 may function as the operating state sensing unit 153. The control unit 140 may control the operating modes of the breath sensor 10 based on the breath sensed by the breath sensor 10. The control unit 140 may control the breath sensor 10 to transition to the standby state when a period during which a magnitude of the breath is equal to or less than a set value continues for or over a set period. In another example, when it is desired to sense the breath in a state where the user 200 is excited, the control unit 140 may control the breath sensor 10 to transition to the standby state when cycles of breathing calculated from the breath are within a set normal range.

Controlling the breath sensor 10 based on a state of the main body 102 as described in FIG. 16 can reduce power consumption of the breath sensor 10. In particular, when no electrical power is supplied from an external power source to the main body 102, the power consumption of the breath sensor 10 can be preferably reduced so that a time period during which the main body 102 can be driven by the storage battery 117 is not shortened.

The control unit 140 may evaluate each state of the main body 102 as described in FIG. 16 or the like as a state value. The state value is a numerical value indicating a degree of a state. For example, the positional state of the movable part 120 can be evaluated by a relative coordinate value of an evaluated position, such as a leading end, of the movable part 120 to a reference position, such as a connector 111, of the fixed part 110 or a rotational angle of the movable part 120 relative to the fixed part 110. The state value may be a value of a temperature, humidity, pressure, or wind speed, may be a magnitude, frequency, or duration of sensing of the sound sensed by the sound sensing unit 122, may be a magnitude, a frequency, a duration of generation of the sound generated by the sound generating unit 112, may be a magnitude of the remaining capacity of the storage battery 117, may be a magnitude of power consumption of the main body 102, or may be a value of another parameter.

The control unit 140 may control the operating modes of the breath sensor 10 by comparing the state value of the main body 102 and the set reference value. The reference value may be set for each type of the state of the main body 102. The type of the state is a type of a parameter such as an angle indicating the state of the main body 102, a magnitude of the sound, or the like.

The reference value for controlling the breath sensor 10 to transition from the active state to the standby state and the reference value for controlling the breath sensor 10 to transition from the standby state to the active state may be the same or may be different. Setting different reference values can prevent transitions between the states of the breath sensor 10 continuously occurring in a short cycle.

The main body 102 has a plurality of types of state and the state of each type may be variable. For example, the main body 102 has a plurality of types of state such as a position of the movable part 120 and a magnitude of the sound sensed by the sound sensing unit 122. Each state such as the position of the movable part 120 and the magnitude of the sound sensed by the sound sensing unit 122 is variable.

The control unit 140 may control the operating modes of the breath sensor 10 based on two or more types of state of the main body 102. The control unit 140 may control the breath sensor 10 to transition to the standby state when at least one of the plurality of states that should be used in control of the operating modes by the breath sensor 10 indicates that the breath sensor 10 should be in the standby state. In other words, the control unit 140 may control the breath sensor 10 to transition to the active state when all of the plurality of states that should be used in the control of the operating modes by the breath sensor 10 indicates that the breath sensor 10 should be in the active state.

In another example, the plurality of types of state of the main body 102 may be categorized into two or more types. For one or more states included in a first category, when at least one state indicates that the breath sensor 10 should be in the standby state, the breath sensor 10 may be controlled to transition to the standby state. For a plurality of states included in a second category, when two or more states indicate that the breath sensor 10 should be in the standby state, the breath sensor 10 may be controlled to transition to the standby state.

The first category may include a state for judging that the breath sensing is not required or power consumption for the breath sensing cannot be allowed. For example, the first category includes a mounted state of the main body 102, a state of power consumption of the main body 102, a state of the remaining capacity of the storage battery 117, a state of a type of the application software, or the like.

The second category may include a state for judging that the accuracy of the breath sensing is lowered. For example, the second category includes a state of a surrounding environment of the breath sensor 10, a state of a position of the movable part 120, a state of the sound in the sound sensing unit 122, a state of the sound in the sound generating unit 112, or the like.

The control unit 140 may perform a process using another state of the second type in the judgment using a state of the first type of the main body 102. For example, the control unit 140 may determine whether the sound sensed by the sound sensing unit 122 is a sound from the user 200 by comparing a temporal waveform of the sound sensed by the sound sensing unit 122 and a temporal waveform of the breath, for example the carbon dioxide concentration, sensed by the breath sensor 10. The breath sensor 10 before the determination is in the active state. The control unit 140 may control the breath sensor 10 to transition from the active state to the standby state when it is determined that a sound from the user 200 is sensed.

The control unit 140 may determine that the sound from the user 200 is sensed, when a correlation coefficient between the temporal waveform of the sound and the temporal waveform of the breath is equal to or higher than a reference value. The correlation coefficient may be represented by a real number from 1 to −1. While the correlation coefficient of 1 indicates that the two temporal waveforms match, the value of the correlation coefficient closer to 0 indicates a lower similarity of the two temporal waveforms. When the user 200 is uttering, carbon dioxide is expelled in accordance with the utterance. Therefore, when the user 200 is uttering, the temporal waveform of the sound and the temporal waveform of the breath often change in synchronism with each other. Therefore, the correlation coefficient increases.

The control unit 140 may determine that the sound from the user 200 is sensed, when a magnitude, for example a peak value, of the sound sensed by the sound sensing unit 122 is equal to or higher than a reference value and also the correlation coefficient between the temporal waveform of the sound and the temporal waveform of the breath is equal to or higher than a reference value. Such a control enables more accurate sensing of the sound from the user 200.

The control unit 140 can perform a process with priorities for categories of the state of the main body 102. The priority for each category may be preset by the user, a manufacturer, or the like. Each category includes the state of the main body 102 of one or more types. Each state of the main body 102 indicates whether the breath sensor 10 should be in the active state or in the standby state.

As an example, a first state of the main body 102 may indicate that the breath sensor 10 should be in the active state, and a second state of the main body 102 may indicate that the breath sensor 10 should be in the standby state. In this manner, when a plurality of states of the main body 102 indicate different operating modes which the breath sensor 10 should be controlled to be in, the control unit 140 may determine into which category each state of the main body 102 is categorized. The control unit 140 may control the operating modes of the breath sensor 10 based on the state of the main body 102 categorized in a category having a highest priority.

An example will be described in which the state of the main body 102 is categorized into three categories. In the present example, a priority of a first category is higher than a priority of a third category. A priority of a second category may be lower than or the same as the priority of the first category. For example, the first category includes a state of the application software. The state of the application software may be a content of an instruction from the application software. For example, the second category includes a state for judging that power consumption for the breath sensing cannot be allowed, a mounted state of the main body 102, a state of the power consumption of the main body 102, a state of the remaining capacity of the storage battery 117, or the like. For example, the third category includes a state for judging that the accuracy of the breath sensing is lowered.

As a more specific example, below described is the application software requesting that the breath sensor 10 be in the active state for adjusting the position of the breath sensor 10. For example, the position of the breath sensor 10 may be adjusted for accurately sensing the breath of the user 200. In this case, the state of the application software categorized into the first category requests the active state. On the other hand, the states of the main body 102 categorized into the second category and the third category are various. For example, when the position of the breath sensor 10 does not allow accurate sensing of the breath, the state of the main body 102 included in the third category requests that the breath sensor 10 be in the standby state. In such a case, the control unit 140 may control the breath sensor 10 to transition to the active state by prioritizing the request from the application software categorized into the first category. Even if the state of the main body 102 categorized into the third category requests that the breath sensor 10 be in the standby state in order to request revision of the surrounding environment, the control unit 140 may maintain the breath sensor 10 in the active state by prioritizing the first category. For example, when a sound is generated due to an impact of wind from an electric fan on the sound sensing unit 122, the state of the main body 102 categorized into the third category requests that the breath sensor 10 be in the standby state. In such a case, the breath sensor 10 can be maintained in the active state in accordance with the request from the application software to prompt the user to avoid the impact of the wind from the electric fan.

As another example, the control unit 140 can perform a process with priorities by combining the plurality of categories. As an example, at least one of the plurality of categories may be an auxiliary category. When the state of the main body 102 categorized into two or more categories indicates that the breath sensor 10 should be controlled to be in different states, the control unit 140 may control the operating modes of the breath sensor 10 based on the state of the main body 102 categorized into the auxiliary category.

As a specific example, the first category includes a state of the application software. The second category includes a state for judging that power consumption for the breath sensing cannot be allowed, a mounted state of the main body 102, a state of the power consumption of the main body 102, or a state of the remaining capacity of the storage battery 117, or the like. The third category includes a state of charging indicating whether the storage battery 117 is being charged. The fourth category includes a state for judging that the accuracy of the breath sensing is lowered. In the present example, the third category is the auxiliary category. Here, assume that the first category requests the active state and the second and fourth categories request the standby state. When requests of other plurality of categories are different from each other, the control unit 140 may control the breath sensor 10 to transition to the active state or to transition to the standby state based on the state of charging in the third category.

The control unit 140 may control the state of the breath sensor 10 based on the state of different types of the main body 102 for when the breath sensor 10 is transitioned from the active state to the standby state and when the breath sensor 10 is transitioned from the standby state to the active state. The control unit 140 may control the breath sensor 10 to transition from the standby state to the active state based on the positional state of the movable part 120. For example, the control unit 140 controls the breath sensor 10 to transition to the active state when the breath sensor 10 is located close to the mouth of the user 200 in accordance with the positional state of the movable part 120. The control unit 140 may control the breath sensor 10 to transition from the active state to the standby state based on the state of the sound sensed by the sound sensing unit 122. For example, the control unit 140 controls the breath sensor 10 to transition from the active state to the standby state when the sound sensing unit 122 senses the utterance of the user 200.

When the breath sensor 10 has three or more states of power consumption, the control unit 140 may control the transition to each operating mode of the breath sensor 10 based on the state of different types of the main body 102. As an example, the breath sensor 10 has three operating modes including a maximum performance state, an eco-drive state, and a standby state. The maximum performance state is an operating mode in which power consumption is maximum and the accuracy or the frequency of the breath sensing is maximum among the three operating modes. The eco-drive state is an operating mode in which power consumption is lower than the maximum performance state and at least one of the accuracy or the frequency of the breath sensing is lower. The standby state is an operating mode in which power consumption is minimal and the accurately or the frequency of the breath sensing is minimal among the three operating modes. The breath sensor 10 in the standby state may not perform the breath sensing.

For example, the control unit 140 may control the breath sensor 10 to transition to any of the operating modes based on the request from the application software. The control unit 140 may control the breath sensor 10 to transition to the eco-drive state when the remaining capacity of the storage battery 117 is lowered while the breath sensor 10 is in the maximum performance state. The control unit 140 may control the breath sensor 10 to transition to the standby state when the remaining capacity of the storage battery 117 is further lowered. The control unit 140 may control the breath sensor 10 to transition from the standby state to the maximum performance state when charging of the storage battery 117 is started in accordance with the state of charging. The control unit 140 may control the breath sensor 10 to transition from the maximum performance state to the standby state when the charging of the storage battery 117 is interrupted to end the charging the battery remaining capacity of the storage battery 117 cannot allow the maximum performance state.

The control unit 140 may adjust the determination criterion of the state of the main body 102 when controlling the operating modes of the breath sensor 10 at a present time, based on the state of the main body 102 used in the determination when controlling the operating modes of the breath sensor 10 at a previous time. The control unit 140 may change the determination criterion for controlling the breath sensor 10 to transition to the standby state between a situation immediately after the movable part 120 is manipulated to move the sound sensing unit 122 to be close to the mouth of the user 200 and other situations.

The user 200 is relatively highly likely to utter during a period immediately after the sound sensing unit 122 is moved to be close to the mouth of the user 200. When the breath sensor 10 transitioned to the active state in accordance with the positional state of the movable part 120 in a previous control, the control unit 140 may lower the reference value to be compared with the magnitude of the sound sensed by the sound sensing unit 122 than in a normal situation. When the magnitude of the sound sensed by the sound sensing unit 122 is greater than the reference value, the control unit 140 determines that the user 200 is uttering. This facilitates sensing of the utterance of the user 200. When having sensed the utterance of the user 200, the control unit 140 controls the breath sensor 10 to transition to the standby state. The normal situation refers to a situation where the breath sensor 10 transitioned to the active state in accordance with a state other than the positional state of the movable part 120 in the previous control.

The control unit 140 may set the reference value to be compared with the remaining capacity of the storage battery 117 higher than in a normal situation when the breath sensor 10 is transitioned to the active state according to the positional state of the movable part 120 in the previous control. The control unit 140 controls the breath sensor 10 to transition to the standby state when the remaining capacity of the storage battery 117 is lower than the reference value. Thus, during a period in which the user 200 is highly likely to utter, the breath can be sensed only when the remaining capacity of the storage battery 117 is ample.

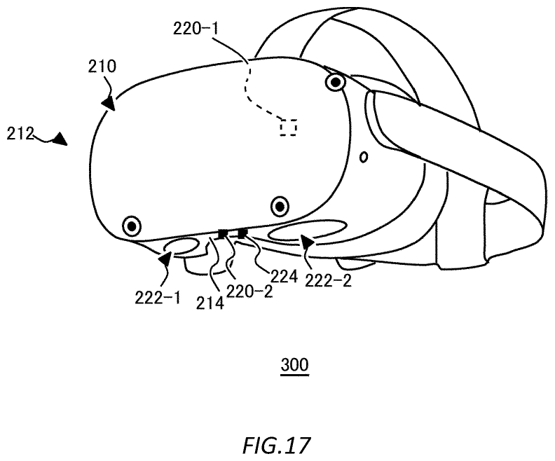

FIG. 17 is a diagram illustrating an example of a display device 300 according to one embodiment of the present invention. The display device 300 includes a head-mounted, or HMD, display unit 210 and a CO₂ (carbon dioxide) sensor 220. The display device 300 including the display unit 210 of the HMD type is, for example, a VR (Virtual Reality) goggle, smart glasses, or the like.

The display device 300 may include a plurality of CO₂ (carbon dioxide) sensors 220. The display device 300 in the present example includes two CO₂ (carbon dioxide) sensor 220, which are CO₂ (carbon dioxide) sensor 220-1 and the CO₂ (carbon dioxide) sensor 220-2. The display device 300 may include an expiratory volume sensor 224.

The CO₂ (carbon dioxide) sensor 220 and the expiratory volume sensor 224 may be included in a housing 212 of the head-mounted, or HMD, display unit 210. The display device 300 in the present example has a plurality of apertures 222, which are an aperture 222-1 and an aperture 222-2, in a bottom surface 214 of the housing 212. In the present example, the CO₂ (carbon dioxide) sensor 220-1 is provided inside of the housing 212 and the CO₂ (carbon dioxide) sensor 220-2 is provided at the bottom surface 214. The CO₂ (carbon dioxide) sensor 220-2 in the present example is exposed to the outside of the housing 212. The expiratory volume sensor 224 may be provided at a surface of the housing 212. The expiratory volume sensor 224 in the present example is provided at the bottom surface 214. When the display device 300 includes a sound acquiring unit which acquires the utterance of the manipulator 310, the expiratory volume sensor 224 may be located at the sound acquiring unit or near the sound acquiring unit.

The CO₂ (carbon dioxide) sensor 220 acquires a CO₂ (carbon dioxide) concentration in the breath of the manipulator 310, which will be described below. In the present example, the CO₂ (carbon dioxide) sensor 220-1 acquires a CO₂ (carbon dioxide) concentration that entered into the housing 212 through the aperture 222 and the CO₂ (carbon dioxide) sensor 220-1 acquires a CO₂ (carbon dioxide) concentration outside of the housing 212. Data of temporal variations of the CO₂ (carbon dioxide) concentration acquired by the CO₂ (carbon dioxide) sensor 220 may be stored in the storage unit 270, which will be described below. Further, the CO₂ (carbon dioxide) sensor 220 may calculate the expiratory volume Vb from the data acquired.

The expiratory volume sensor 224 acquires an expiratory volume Vb, which will be described below, of the manipulator 310, which will be described below. The expiratory volume sensor 224 in the present example acquires the expiratory volume Vb entered into the housing 212 through the aperture 222. Data of temporal variations of the expiratory volume Vb, which will be described below, acquired by the expiratory volume sensor 224 may be stored in the storage unit 270, which will be described below.

The CO₂ (carbon dioxide) sensor 220 and the expiratory volume sensor 224 may acquire an expiratory cycle of the manipulator 310, which will be described below. The CO₂ (carbon dioxide) sensor 220 and the expiratory volume sensor 224 may acquire an expiratory volume Vb per breath, which will be described below, based on the expiratory cycle,

For example, when the CO₂ (carbon dioxide) sensor 220 detects a certain level of carbon dioxide concentration, the expiratory volume Vb tends to increase as the breathing cycle is longer and the expiratory volume Vb tends to decrease as the expiratory cycle is shorter.

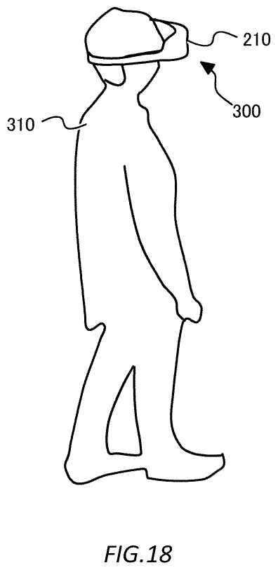

FIG. 18 is a diagram illustrating an example of the manipulator 310 of the display unit 210. FIG. 18 omits the CO₂ (carbon dioxide) sensor 220 and the expiratory volume sensor 224 illustrated in FIG. 17. The manipulator 310 in the present example is wearing the display device 300. The manipulator 310 is a living body. The manipulator 310 in the present example is a human. The manipulator 310 visually recognizes a character, an image, or the like displayed on the display unit 210. The manipulator 310 may visually recognize an image or the like of a virtual reality space displayed on the display unit 210.

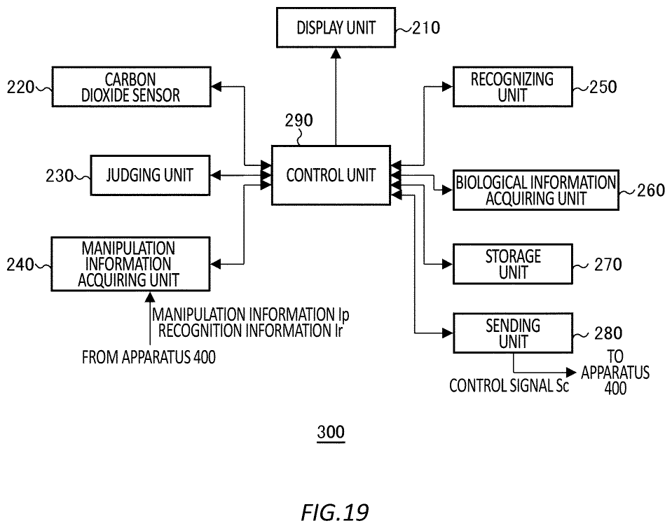

FIG. 19 is a block diagram showing an example of the display device 300 according to one embodiment of the present invention. The display device 300 may include a judging unit 230, a manipulation information acquiring unit 240, a recognizing unit 250, a biological information acquiring unit 260, a storage unit 270, a sending unit 280, and a control unit 290.

A part or an entirety of the display device 300 or the breath sensing system 100 as shown in FIG. 13 may be implemented by a computer. The control unit 290 may be a central processing unit (CPU) of this computer.

When the display device 300 is implemented by a computer, a display program which causes this computer to function as the display device 300 may be installed in this computer, or a display program which causes this computer to execute a display method described below may be installed in this computer.

The judging unit 230 judges a state of the manipulator 310 based on CO₂ (carbon dioxide) concentration acquired by the CO₂ (carbon dioxide) sensor 220. The state of the manipulator 310 is referred to as a state S. The state S of the manipulator 310 may refer to a state of action or a state of emotion of the manipulator 310. For example, the state of action of the manipulator 310 indicates whether the manipulator 310 is in a resting state or in an exercising state, or the like. When the manipulator 310 is in the exercising state, the state of action of the manipulator 310 indicates, for example, an anaerobic exercise state or an aerobic exercise state. The state of emotion of the manipulator 310 indicates, for example, the manipulator 310 is in an unrelaxed state or in a relaxed state, or the like. Whether the manipulator 310 is in the resting state or in the exercising state may be judged by whether the CO₂ (carbon dioxide) concentration in the breath is equal to or higher than a predetermined threshold Cth, which will be described below or may be judged by whether the expiratory volume Vb, which will be described below, is equal to or higher than a predetermined threshold Vth, which will be described below.

The display unit 210 may display the state S of the manipulator 310 judged by the judging unit 230. The CO₂ (carbon dioxide) concentration in the breath of the manipulator 310 tends to vary in accordance with the state S of the manipulator 310. Thus, the manipulator 310 can recognize his/her own state S by the display unit 210 displaying the state S.

The control unit 290 may control the display unit 210 based on the state S of the manipulator 310 judged by the judging unit 230. Controlling the display unit 210 based on the state S by the control unit 290 may include controlling the display unit 210 to display text information according to the state S and may include controlling a brightness, coloring, or the like of the display unit 210 based on the state S by the control unit 290. For example, the text information according to the state S is text information such as “Aerobic exercise state” indicating that the manipulator 310 is in the aerobic exercise state.

The control unit 290 may control the display unit 210 based on a CO₂ (carbon dioxide) concentration acquired by the CO₂ (carbon dioxide) sensor 220 and the reference value of the CO₂ (carbon dioxide) concentration. The reference value of the CO₂ (carbon dioxide) concentration is referred to as a reference value Vs. The reference value Vs is a CO₂ (carbon dioxide) concentration corresponding to the state S of the manipulator 310. As described above, the CO₂ (carbon dioxide) concentration in the breath of the manipulator 310 tends to vary in accordance with the state S of the manipulator 310.

The reference value Vs may be a CO₂ (carbon dioxide) concentration in the breath of the manipulator 310 defined for each of a plurality of state S. The reference value Vs in one state S of the manipulator 310 and the reference value Vs in another state S of the manipulator 310 may be different. The reference value Vs for each state S may be stored in the storage unit 270.

The reference value Vs may be defined for each of a plurality of manipulators 310. The reference value Vs in one state S, for example an unrelaxed state, of one manipulator 310 and the reference value Vs in the one state S, for example an unrelaxed state, of another manipulator 310 may be different. The reference value Vs for each manipulator 310 may be stored in the storage unit 270.

The control unit 290 may control the display unit 210 so that the CO₂ (carbon dioxide) concentration acquired by the CO₂ (carbon dioxide) sensor 220 comes closer to the reference value Vs. For example, when the state S of the manipulator 310 is judged to be the anaerobic exercise state, the control unit 290 controls the display unit 210 to display a phrase such as “Let's slow down your pace a little more” on the display unit 210 so that the CO₂ (carbon dioxide) concentration in the breath of the manipulator 310 comes closer to the reference value Vs for an aerobic exercise. For example, when the manipulator 310 is judged to be in an unrelaxed state, the control unit 290 controls the display unit 210 to change the image displayed on the display unit 210 to a bright color so that the CO₂ (carbon dioxide) concentration in the breath of the manipulator 310 comes closer to the reference value Vs for a relaxed state.

The CO₂ (carbon dioxide) sensor 220 may further acquire the CO₂ (carbon dioxide) concentration in the breath of the manipulator 310 after the control unit 290 controlled the display unit 210 based on the CO₂ (carbon dioxide) concentration and the reference value Vs. The judging unit 230 may judge the state S of the manipulator 310 based on the CO₂ (carbon dioxide) concentration after the display unit 210 is controlled. The display unit 210 may display the state S. Thus, the manipulator 310 can check whether his/her own state S is in a desired state S.

The control unit 290 may control the display unit 210 based on a change from the CO₂ (carbon dioxide) concentration before the display unit 210 is controlled to the CO₂ (carbon dioxide) concentration after the display unit 210 is controlled. The judging unit 230 may judge the state S of the manipulator 310 after the display unit 210 is controlled. The control unit 290 may control the display unit 210 based on the state S of the manipulator 310. Thus, the manipulator 310 can check the display unit 210 is controlled so that his/her own state S is in the desired state S.

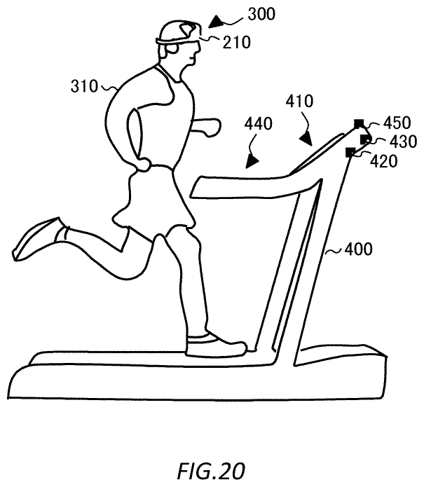

FIG. 20 is a diagram illustrating an example of a situation where the manipulator 310 using the apparatus 400. The apparatus 400 may affect an increase and a decrease in the CO₂ (carbon dioxide) concentration in the breath of the manipulator 310. For example, the apparatus 400 is an apparatus for exercise, i.e., an exercise machine or a controller manipulated by the manipulator 310 for manipulating the gaming device in an experience-based gaming device, or the like. The apparatus 400 in the present example is a treadmill.

The manipulator 310 may use the apparatus 400 while wearing the display device 300. FIG. 20 omits the CO₂ (carbon dioxide) sensor 220 included in the display device 300. The CO₂ (carbon dioxide) concentration in the breath of the manipulator 310 may change in accordance with a use situation of the apparatus 400 by the manipulator 310. When the apparatus 400 is an apparatus for exercise, the experience-based gaming device, or the like, the state of the manipulator 310 tends to change in accordance with the use situation of the apparatus 400. For example, when the apparatus 400 is the treadmill, the CO₂ (carbon dioxide) concentration in the breath of the manipulator 310 or the expiratory volume Vb, which will be described below, or the expiratory cycle tends to change in accordance with use time of the treadmill by the manipulator 310. For example, when the apparatus 400 is the experience-based gaming device, the manipulator 310 may fall into an excited state while playing the game. When the manipulator 310 has fallen into the excited state, the manipulator 310 may move his/her own body. Thus, the CO₂ (carbon dioxide) concentration in the breath of the manipulator 310, or the expiratory volume Vb, which will be described below, or the expiratory cycle may change in accordance with the use situation of the experience-based gaming apparatus by the manipulator 310.

The apparatus 400 may include a display unit 410, a transceiver unit 420, a control unit 430, a manipulation unit 440, and a recognizing unit 450. The control unit 430 may be a CPU. The display unit 410 is, for example, a display, a monitor, or the like. The manipulation unit 440 is, for example, a button, a keyboard, or the like. The recognizing unit 450 is, for example, a photographing device such as a camera. The manipulator 310 performs a manipulation such as setting of the apparatus 400 by using the manipulation unit 440, or the like.

The control unit 290 may control the apparatus 400 based on the state S of the manipulator 310 judged by the judging unit 230. The control unit 290 may generate a control signal Sc as shown in FIG. 19 for controlling the apparatus 400. The sending unit 280 as shown in FIG. 19 may send the control signal Sc generated to the transceiver unit 420 of the apparatus 400. The sending unit 280 may wirelessly send the control signal Sc to the transceiver unit 420. The transceiver unit 420 may receive the control signal Sc. The control unit 430 may control the display unit 410 based on the control signal Sc and may control the apparatus 400.

Controlling the apparatus 400 based on the state S by the control unit 290 may include controlling the display unit 410 to display text information according to the state S and may include controlling a brightness, coloring, or the like of the display unit 410 based on the state S by the control unit 290. For example, the text information according to the state S is text information such as “Aerobic exercise state” indicating that the manipulator 310 is in the aerobic exercise state.

The control unit 290 may control the apparatus 400 based on the CO₂ (carbon dioxide) concentration in the breath of the manipulator 310 and the reference value Vs of CO₂ (carbon dioxide) concentration.

The control unit 290 may control the apparatus 400 so that the CO₂ (carbon dioxide) concentration acquired by the CO₂ (carbon dioxide) sensor 220 comes closer to the reference value Vs. For example, when the state S of the manipulator 310 is judged to be the anaerobic exercise state, the control unit 290 controls the display unit 410 to display a phrase such as “Let's slow down your pace a little more” on the display unit 410 so that the CO₂ (carbon dioxide) concentration in the breath of the manipulator 310 comes closer to the reference value Vs for an aerobic exercise. For example, if the manipulator 310 is playing a game using the experience-based gaming device, when the state S of the manipulator 310 is judged to be the unrelaxed state, the control unit 290 controls a screen of the gaming device so that the state S of the manipulator 310 comes close to the reference value Vs of the relaxed state or daringly controls the screen of the gaming device so that the state S of the manipulator 310 comes close to the reference value Vs of excessively unrelaxed state.

The CO₂ (carbon dioxide) sensor 220 may further acquire the CO₂ (carbon dioxide) concentration in the breath of the manipulator 310 after the control unit 290 controlled the apparatus 400 based on the CO₂ (carbon dioxide) concentration and the reference value Vs. The judging unit 230 may judge the state S of the manipulator 310 based on the CO₂ (carbon dioxide) concentration after the apparatus 400 is controlled. The display unit 410 may display the state S. Thus, the manipulator 310 can check whether his/her own state S is in a desired state S.

The control unit 290 may control the apparatus 400 based on a change from the CO₂ (carbon dioxide) concentration before the apparatus 400 is controlled to the CO₂ (carbon dioxide) concentration after the apparatus 400 is controlled. The judging unit 230 may judge the state S of the manipulator 310 after the apparatus 400 is controlled. The control unit 290 may control the apparatus 400 based on the state S of the manipulator 310. Thus, the manipulator 310 can check the apparatus 400 is controlled so that his/her own state S is in the desired state S.

The manipulation information acquiring unit 240 as shown in FIG. 19 acquires manipulation information of the apparatus 400 by the manipulator 310. The manipulation information is referred to as manipulation information Ip. For example, if the apparatus 400 is an exercise machine that brings the manipulator 310 into the exercising state by operation of the apparatus 400, the manipulation information Ip is information about how the manipulator 310 manipulated the operating state of the apparatus 400. For example, the manipulation information Ip is information indicating that the operating state of the exercise machine is intensified or weakened. For example, intensifying the operating state of the exercise machine means that the manipulator 310 performing a manipulation for increasing input power to the exercise machine. For example, weakening the operating state of the exercise machine means that the manipulator 310 performing a manipulation for decreasing the input power to the exercise machine.

For example, if the apparatus 400 is a treadmill, when the manipulator 310 feels that the speed of the moving strap of the treadmill is high, the manipulator 310 may manipulate the apparatus 400 by the manipulation unit 440 so as to decrease the speed of the moving strap. In this case, the manipulation information Ip is information indicating that the manipulator 310 manipulated to decrease the speed of the moving strap.

The judging unit 230 as shown in FIG. 19 may judge the state S of the manipulator 310 based on the CO₂ (carbon dioxide) concentration in the breath of the manipulator 310 and the manipulation information Ip. The manipulation information Ip may reflect the state S of the manipulator 310. Thus, the judging unit 230 may judge the state S of the manipulator 310 based on the manipulation information Ip. If the apparatus 400 is a treadmill, the judging unit 230 may judge the state S related to a load of the exercise of the manipulator 310 based on the manipulation information Ip.

The control unit 290 may control the apparatus 400 based on the state S that is judged. For example, if the apparatus 400 is a treadmill, when the state S of the manipulator 310 is judged to be the anaerobic exercise state, the control unit 290 controls the apparatus 400 so as to increase the speed of the moving strap of the treadmill so that the CO₂ (carbon dioxide) concentration in the breath of the manipulator 310 matches with a concentration defined for the aerobic exercise.

If the apparatus 400 is an exercise machine, the display unit 210 may display a reaction to the manipulation of the apparatus 400 by the manipulator 310 based on the state S of the manipulator 310 based on the CO₂ (carbon dioxide) concentration judged by the judging unit 230 and the manipulation information Ip. For example, when the state S is judged to be the exercising state and the manipulation information Ip is information indicating that the operating state of the exercise machine is intensified, the display unit 210 displays, to the manipulator 310, a message such as “Keep it up!” for example as a reaction to the manipulation of the apparatus 400. For example, when the state S is judged to be the exercising state and the manipulation information Ip is information indicating that the operating state of the exercise machine is weakened, the display unit 210 displays, to the manipulator 310, a message such as “Good job!” for example as a reaction to the manipulation of the apparatus 400. For example, when the state S is judged to be the resting state and the manipulation information Ip is information indicating that the operating state of the exercise machine is intensified, the display unit 210 displays, to the manipulator 310, a message such as “Let's get started” for example as a reaction to the manipulation of the apparatus 400. For example, when the state S is judged to be the resting state and the manipulation information Ip is information indicating that the operating state of the exercise machine is weakened, the display unit 210 displays, to the manipulator 310, a message such as “Would you like to exercise?” for example as a reaction to the manipulation of the apparatus 400.

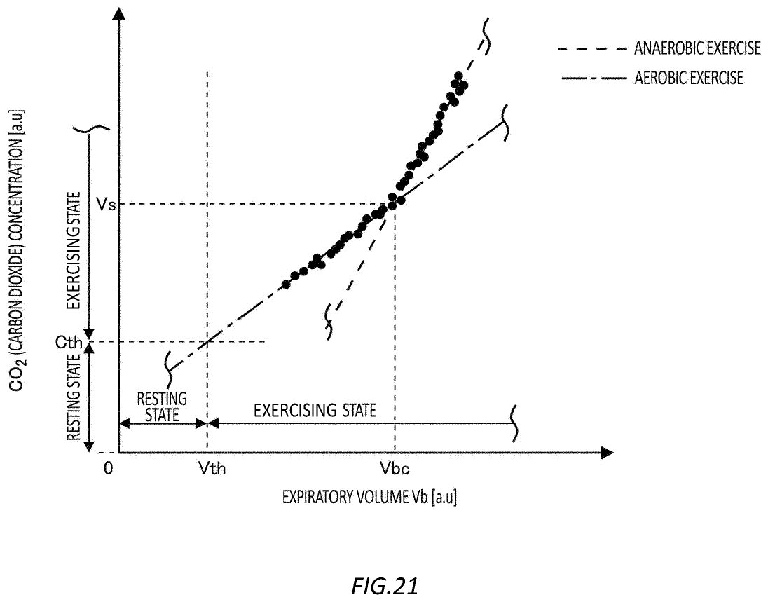

FIG. 21 is a diagram illustrating an example of a relationship between a CO₂ (carbon dioxide) concentration in the breath of the manipulator 310 and an expiratory volume of the manipulator 310. The expiratory volume is referred to as an expiratory volume Vb. The expiratory volume sensor 224 as shown in FIG. 17 acquires the expiratory volume Vb. The judging unit 230 may judge the state S of the manipulator 310 based on the CO₂ (carbon dioxide) concentration and the expiratory volume Vb. The judging unit 230 may judge that the manipulator 310 is in the exercising state when the expiratory volume Vb is equal to or higher than a predetermined threshold Vth. The judging unit 230 may judge that the manipulator 310 is in the resting state when the expiratory volume Vb is lower than threshold Vth. The judging unit 230 may judge that the manipulator 310 is in the exercising state when the CO₂ (carbon dioxide) concentration in the breath is equal to or higher than a predetermined threshold Cth. The judging unit 230 may judge that the manipulator 310 is in the resting state when the CO₂ (carbon dioxide) concentration is lower than the threshold Cth. The Vs of the CO₂ (carbon dioxide) concentration in FIG. 21 is the reference value Vs as described above when the manipulator 310 is in the exercising state.

The judging unit 230 as shown in FIG. 19 may judge whether the manipulator 310 is in the anaerobic exercise state or in the aerobic exercise state based on a predetermined relationship between the CO₂ (carbon dioxide) concentration in the breath of the manipulator 310 and the expiratory volume Vb. For example, the predetermined relationship between the CO₂ (carbon dioxide) concentration in the breath and the expiratory volume Vb is a relationship shown in FIG. 21.

As described above, the expiratory volume Vb per breath, which will be described below, tends to increase as the expiratory cycle is longer and tends to decrease as the expiratory cycle is shorter. Thus, the judging unit 230 as shown in FIG. 19 may judge whether the manipulator 310 is in the anaerobic exercise state or in the aerobic exercise state based on the predetermined relationship between the CO₂ (carbon dioxide) concentration in the breath of the manipulator 310 and the expiratory cycle.

In FIG. 21, the anaerobic exercise state is indicated by a rough dashed line and the aerobic exercise state is indicated by a one-dot chain line. The CO₂ (carbon dioxide) concentration tends to increase as the expiratory volume Vb increases. When the expiratory volume Vb is equal to or higher than the expiratory volume Vbc, the manipulator 310 tends to be in the anaerobic exercise state. When the expiratory volume Vb is lower than the expiratory volume Vbc, the manipulator 310 tends to be in the aerobic exercise state. When the CO₂ (carbon dioxide) concentration in the breath is equal to or higher than the reference value Vs, the manipulator 310 tends to be in the anaerobic exercise state. When the CO₂ (carbon dioxide) concentration in the breath is lower than the reference value Vs, the manipulator 310 tends to be in the aerobic exercise state. An increase rate of the CO₂ (carbon dioxide) concentration per unit of expiratory volume when performing an anaerobic exercise tends to be higher than an increase rate of the CO₂ (carbon dioxide) concentration per unit of expiratory volume when performing an aerobic exercise.

The judging unit 230 as shown in FIG. 19 may judge whether the manipulator 310 is in the anaerobic exercise state or in the aerobic exercise state based on the expiratory volume Vbc. The judging unit 230 may judge that the manipulator 310 is in the anaerobic exercise state when the expiratory volume Vb is equal to or higher than the expiratory volume Vbc. The judging unit 230 may judge that the manipulator 310 is in the aerobic exercise state when the expiratory volume Vb is lower than the expiratory volume Vbc. The judging unit 230 as shown in FIG. 19 may judge whether the manipulator 310 is in the anaerobic exercise state or in the aerobic exercise state based on the expiratory cycle corresponding to the expiratory volume Vbc.

The judging unit 230 as shown in FIG. 19 may judge whether the manipulator 310 is in the anaerobic exercise state or in the aerobic exercise state based on the reference value Vs. When the CO₂ (carbon dioxide) concentration in the breath is equal to or higher than the reference value Vs, the judging unit 230 may judge that the manipulator 310 is in the anaerobic exercise state. When the CO₂ (carbon dioxide) concentration in the breath is lower than the reference value Vs, the judging unit 230 may judge that the manipulator 310 is in the aerobic exercise state.

The recognizing unit 250 as shown in FIG. 19 recognizes the manipulator 310. The recognizing unit 250 may be included in the housing of the head-mounted, or HMD, display unit 210. For example, the recognizing unit 250 recognizes the manipulator 310 by recognizing a face of the manipulator 310. The recognizing unit 250 may recognize one particular manipulator 310 among a plurality of manipulators 310.

The recognizing unit 450 as shown in FIG. 20 recognizes the manipulator 310. The recognizing unit 450 may recognize the manipulator 310 by recognizing the face of the manipulator 310. The recognizing unit 450 may recognize one particular manipulator 310 among a plurality of manipulators 310. The control unit 430 as shown in FIG. 20 may generate recognition information of the manipulator 310 recognized by the recognizing unit 450. The recognition information is referred to as recognition information Ir.

The transceiver unit 420 as shown in FIG. 20 may send the recognition information Ir. The transceiver unit 420 may wirelessly send the recognition information Ir. The manipulation information acquiring unit 240 as shown in FIG. 19 may acquire the recognition information Ir. The recognizing unit 250 as shown in FIG. 19 may recognize the manipulator 310 based on the recognition information Ir.

The relationship between the CO₂ (carbon dioxide) concentration in the breath and the expiratory volume Vb or the expiratory cycle may be predetermined for each of the plurality of manipulators 310. The relationship between the CO₂ (carbon dioxide) concentration in the breath and the expiratory volume Vb or the expiratory cycle may be different for each of the manipulators 310. The relationship between the CO₂ (carbon dioxide) concentration in the breath of each of the plurality of manipulators 310 and the expiratory volume Vb or the expiratory cycle may be stored in the storage unit 270 as shown in FIG. 19.

The judging unit 230 as shown in FIG. 19 may judge whether the manipulator 310 is in the anaerobic exercise state or in the aerobic exercise state based on the relationship between the CO₂ (carbon dioxide) concentration in the breath and the expiratory volume Vb or the expiratory cycle regarding the manipulator 310 recognized by the recognizing unit 250. Thus, the judging unit 230 may judge, for each of the manipulators 310, whether the manipulator 310 is in the anaerobic exercise state or in the aerobic exercise state.

When the expiratory volume Vb is lower than the threshold Vth, the judging unit 230 as shown in FIG. 19 may judge the emotion of the manipulator 310 based on the CO₂ (carbon dioxide) concentration in the breath. When the expiratory volume Vb is lower than the threshold Vth, the manipulator 310 is in the resting state, as described above. When the expiratory volume Vb is lower than the threshold Vth, the CO₂ (carbon dioxide) concentration in the breath of the manipulator 310 may vary in accordance with the emotion of the manipulator 310. If the expiratory volume Vb is lower than the threshold Vth, the judging unit 230 may judge that the state S of the manipulator 310 is the unrelaxed state when the CO₂ (carbon dioxide) concentration in the breath of the manipulator 310 is equal to or higher than the threshold Dth and the judging unit 230 may judge that the state S of the manipulator 310 is the relaxed state when the CO₂ (carbon dioxide) concentration is lower than the threshold Dth.

The biological information acquiring unit 260 as shown in FIG. 19 acquires biological information of the manipulator 310. The biological information is referred to as biological information Ig. The biological information Ig may include at least one of heartbeat information, perspiration amount information, body temperature information, or electromyography information of the manipulator 310. The biological information acquiring unit 260 may be included in the housing of the head-mounted, or HMD, display unit 210.

When the expiratory volume Vb is lower than the threshold Vth, the judging unit 230 as shown in FIG. 19 may judge the emotion of the manipulator 310 based on the CO₂ (carbon dioxide) concentration in the breath and the biological information Ig. For example, when the manipulator 310 is in the unrelaxed state, the manipulator 310 tends to be in the state in which the sympathetic nervous system is dominant over the parasympathetic nervous system. When the sympathetic nervous system is dominant over the parasympathetic nervous system, a heart rate variation of the manipulator 310 tends to be smaller. For example, when the manipulator 310 is in the relaxed state, the manipulator 310 tends to be in a state in which the parasympathetic nervous system is dominant over the sympathetic nervous system. When the parasympathetic nervous system is dominant over the sympathetic nervous system, the heart rate variation of the manipulator 310 tends to be larger.

A magnitude of a first power spectrum in the heartbeat of the manipulator 310 is referred to as LF and a magnitude of a second power spectrum is referred to as HF. A frequency band of the second power spectrum is a band in which a frequency is higher than that in a frequency band of the first power spectrum. For example, the frequency band of the first power spectrum is 0.05 Hz or more and 0.20 Hz or less. For example, the frequency band of the second power spectrum is 0.20 Hz or more and 0.35 Hz or less.

The biological information acquiring unit 260 as shown in FIG. 19 may acquire an LF and an HF in a heartbeat of the manipulator 310. When a ratio of the LF to the HF, or LF/HF, is equal to or higher than a predetermined threshold Nth, the judging unit 230 as shown in FIG. 19 may judge that the state S of the manipulator 310 is a state in which the parasympathetic nervous system is dominant over the sympathetic nervous system. When a ratio of the LF to the HF, or LF/HF, is lower than the threshold Nth, the judging unit 230 may judge that the state S of the manipulator 310 is a state in which the sympathetic nervous system is dominant over the parasympathetic nervous system.

When the state S of the manipulator 310 is the state in which the sympathetic nervous system is dominant over the parasympathetic nervous system, the expiratory volume Vb of the manipulator 310 tends to increase or the expiratory cycle tends to be longer. When the state S of the manipulator 310 is the state in which the parasympathetic nervous system is dominant over the sympathetic nervous system, the expiratory volume Vb of the manipulator 310 tends to decrease or the expiratory cycle tends to be shorter.

When the sympathetic nervous system is dominant over the parasympathetic nervous system because the manipulator 310 is in the unrelaxed state or a scared state, the manipulator 310 may be in a state of respiratory alkalosis. The state of respiratory alkalosis is a state in which CO₂ (carbon dioxide) is excessively expelled due to hyperventilation or the like. When the manipulator 310 is in the state of respiratory alkalosis, the manipulator 310 tends to experience a rise in the heart rate, the perspiration amount, and the respiratory rate, stiffening of muscles, a fall of the body temperature which may be the body temperature at hands and legs in particular, and a fall of the end-tidal carbon dioxide concentration. In other words, when in the unrelaxed state or the scared state, the manipulator 310 tends to experience an increase in the heart rate, the perspiration amount, and the respiratory rate, and an increase in an amplitude of myogenic potential, and a fall of the CO₂ (carbon dioxide) concentration in the breath.

When the manipulator 310 is in an excited state, an angry state, or a highly aggressive state, the manipulator 310 may be in the state of respiratory acidosis. The state of respiratory acidosis is a state in which the CO₂ (carbon dioxide) is accumulated due to a poor breathing state. When in the state of respiratory acidosis, the manipulator 310 tends to experience a rise in the body temperature which may be the body temperature of the head, chest, or hands in particular and a rise in the end-tidal carbon dioxide concentration. In other words, when in the excited state, the angry state, or the highly aggressive state, the manipulator 310 tends to experience an increase in the heart rate and the perspiration amount, a decrease in the respiratory rate and the respiratory volume Vb and a rise in the CO₂ (carbon dioxide) concentration in the breath.

The judging unit 230 may judge whether the expiratory volume Vb of the manipulator 310 is equal to or higher than the threshold Vth or lower than the threshold Vth based on the biological information Ig. The judging unit 230 may judge the emotion of the manipulator 310 based on the expiratory volume Vb judged and the CO₂ (carbon dioxide) concentration in the breath.

The judging unit 230 as shown in FIG. 19 may judge the application being activated by the display device 300. The judging unit 230 may judge a type of the application being activated by the display device 300. The judging unit 230 may judge the state S of the manipulator 310 based on the application that is judged. For example, if the application activated by the display device 300 is an application related to an exercise such as an application for running, walking, or the like, the state S of the manipulator 310 is probably in the exercising state. For example, if the application activated by the display device 300 is an application unrelated to exercise such as an application for a go, shogi, chess, or the like, the state S of the manipulator 310 is probably in the resting state. Thus, the judging unit 230 can judge the state S of the manipulator 310 based on the application.

The judging unit 230 may judge a type of the application being activated by the display device 300. For example, the type of the application indicates that whether the application is highly related to exercise such as an application for running, walking, or the like, barely related to exercise such as an application for go, shogi, chess, or the like. When the manipulator 310 is using the application highly related to exercise, the manipulator 310 is highly likely to be in the exercising state. When the manipulator 310 is using the application barely related to exercise, the manipulator 310 is highly likely to be in the resting state. The judging unit 230 may estimate the state of the manipulator 310 based on the type of the application that is judged. The judging unit 230 may estimate whether the state of the manipulator 310 is the exercising state or the resting state based on the type of the application that is judged.

The judging unit 230 may judge whether the state S of the manipulator 310 based on the CO₂ (carbon dioxide) concentration acquired by the CO₂ (carbon dioxide) sensor 220 and the state of the manipulator 310 estimated based on the type of the application match. When the state S of the manipulator 310 based on the CO₂ (carbon dioxide) concentration and the state of the manipulator 310 estimated based on the type of the application do not match, the display unit 210 may display information for prompting to quit the activated application. For example, when the state S based on the CO₂ (carbon dioxide) concentration is the exercising state although the state estimated based on the type of the application is the resting state, the manipulator 310 may be playing a go game on his/her smartphone, for example, while walking. This behavior of the manipulator 310 may bother the people around. Thus, the display unit 210 may display information for prompting to quit the activated application.

For example, when the application is an application which acquires workout information such as an application for running or the like, the judging unit 230 may judge the application is an application highly related to exercise. The judging unit 230 may estimate that the state of the manipulator 310 is the exercising state based on the type of the application. When the state S based on the CO₂ (carbon dioxide) concentration is the resting state although the judging unit 230 estimated that the state of the manipulator 310 is the exercising state based on the type of the application, the manipulator 310 may be not sufficiently performing the exercise. Therefore, the display unit 210 may display information for the manipulator 310 to start to exercise or information for prompting the manipulator 310 to exercise.

The CO₂ (carbon dioxide) concentration acquired by the CO₂ (carbon dioxide) sensor 220 may be computed in the control unit 290 as shown in FIG. 19 or may be computed in the application installed in the display device 300. The CO₂ (carbon dioxide) concentration acquired by the CO₂ (carbon dioxide) sensor 220 may be sent from the sending unit 280 as shown in FIG. 19 to the apparatus 400 and the transceiver unit 420 as shown in FIG. 20 may receive the CO₂ (carbon dioxide) concentration. The control unit 430 as shown in FIG. 20 may compute the CO₂ (carbon dioxide) concentration. The transceiver unit 420 as shown in FIG. 20 may send the CO₂ (carbon dioxide) concentration that is computed. The manipulation information acquiring unit 240 as shown in FIG. 19 may receive the CO₂ (carbon dioxide) concentration sent from the transceiver unit 420. The judging unit 230 may judge the state S of the manipulator 310 based on the manipulation information Ip as shown in FIG. 19 and the CO₂ (carbon dioxide) concentration acquired by the manipulation information acquiring unit 240.

When the display device 300 includes a plurality of CO₂ (carbon dioxide) sensors 220, one CO₂ (carbon dioxide) sensor 220 may acquire the CO₂ (carbon dioxide) concentration in the breath accompanied by the utterance of the manipulator 310 and another CO₂ (carbon dioxide) sensor 220 may acquire the CO₂ (carbon dioxide) concentration in the breath unaccompanied by the utterance of the manipulator 310. In the example of FIG. 17, the CO₂ (carbon dioxide) sensor 220-2 acquires the CO₂ (carbon dioxide) concentration in the breath accompanied by the utterance of the manipulator 310 and the CO₂ (carbon dioxide) sensor 220-1 acquires the CO₂ (carbon dioxide) concentration in the breath unaccompanied by the utterance of the manipulator 310.

The display device 300 may include a sound acquiring unit which acquires the utterance of the manipulator 310. In the example of FIG. 17, the CO₂ (carbon dioxide) sensor 220-2 may include the sound acquiring unit. The CO₂ (carbon dioxide) sensor 220-2 may be located closer to the mouth of the manipulator 310 than the CO₂ (carbon dioxide) sensor 220-1. The CO₂ (carbon dioxide) sensor 220-1 may be located farther from the mouth of the manipulator 310 than the CO₂ (carbon dioxide) sensor 220-2. In the example of FIG. 17, the CO₂ (carbon dioxide) sensor 220-1 is located above the head of the manipulator 310.

The CO₂ (carbon dioxide) sensor 220-1 may be separated from the display device 300 oy may be installed in the room in which the manipulator 310 is present. When the CO₂ (carbon dioxide) sensor 220-1 is installed in the room in which the manipulator 310 is present, the CO₂ (carbon dioxide) concentration measured by the CO₂ (carbon dioxide) sensor 220-1 may be wirelessly sent to the display device 300.

The judging unit 230 as shown in FIG. 19 may judge the state S of the manipulator 310 based on the CO₂ (carbon dioxide) concentration acquired by the one CO₂ (carbon dioxide) sensor 220 and the CO₂ (carbon dioxide) concentration acquired by the another CO₂ (carbon dioxide) sensor 220. A difference between the CO₂ (carbon dioxide) concentration acquired by the one CO₂ (carbon dioxide) sensor 220 and the CO₂ (carbon dioxide) concentration acquired by the another CO₂ (carbon dioxide) sensor 220 is referred to as a difference d. The judging unit 230 may judge the state S of the manipulator 310 based on the difference d.

When the difference d is equal to or higher than a threshold dth, the judging unit 230 may judge that the state S of the manipulator 310 is the excited state. When the manipulator 310 is excited, the difference d tends to be larger. When the difference d is lower than the threshold dth, the judging unit 230 may judge that the state S of the manipulator 310 is the relaxed state. When the manipulator 310 is relaxed, the difference d tends to be smaller.

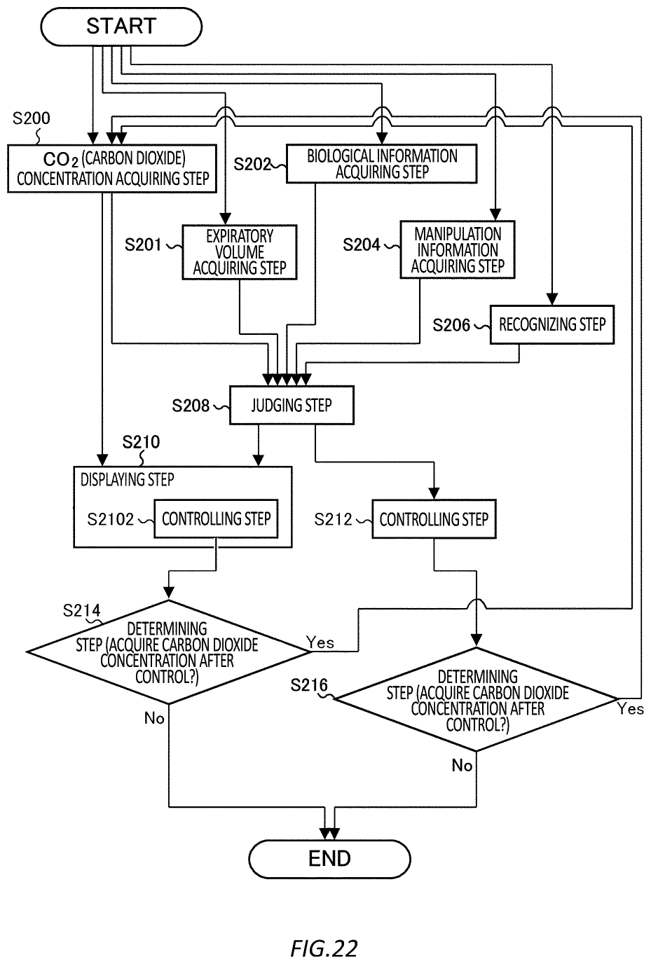

FIG. 22 is a flowchart illustrating an example of a display method according to one embodiment of the present invention. The display method according to one embodiment of the present invention will be described with reference to an example of the display device illustrated in FIG. 19. The display method includes a CO₂ (carbon dioxide) concentration acquiring step S200 and a displaying step S210. The display method may include an expiratory volume acquiring step S201, a biological information acquiring step S202, a manipulation information acquiring step S204, a recognizing step S206, a judging step S208, a controlling step S2102, a controlling step S212, a determining step S214, and a determining step S216.

In the CO₂ (carbon dioxide) concentration acquiring step S200, the CO₂ (carbon dioxide) sensor 220 acquires the CO₂ (carbon dioxide) concentration in the breath of the manipulator 310 manipulating the head-mounted display unit 210. In the displaying step S210, the display unit 210 displays the state S of the manipulator 310 based on the CO₂ (carbon dioxide) concentration.

In the judging step S208, the judging unit 230 may judge the state S of the manipulator 310 based on the CO₂ (carbon dioxide) concentration acquired in the CO₂ (carbon dioxide) concentration acquiring step S200. In the displaying step S210, the display unit 210 may display the state S of the manipulator 310 based on the CO₂ (carbon dioxide) concentration judged by the judging step S208.

The displaying step S210 may include a controlling step S2102. In the controlling step S2102, the control unit 290 may control the display unit 210 based on the state S of the manipulator 310 judged in the judging step S208. In the controlling step S2102, the control unit 290 may control the display unit 210 based on the CO₂ (carbon dioxide) concentration acquired in the CO₂ (carbon dioxide) concentration acquiring step S200 and the reference value Vs of the CO₂ (carbon dioxide) concentration.

In the determining step S214, the judging unit 230 determines whether to acquire the CO₂ (carbon dioxide) concentration after the controlling step S2102. When it is determined to acquire the CO₂ (carbon dioxide) concentration, the display method returns to the CO₂ (carbon dioxide) concentration acquiring step S200. When it is determined not to acquire the CO₂ (carbon dioxide) concentration, the display method ends.

After the display unit 210 is controlled based on the CO₂ (carbon dioxide) concentration and the reference value Vs in the controlling step S2102, the CO₂ (carbon dioxide) sensor 220 may further acquire the CO₂ (carbon dioxide) concentration in the CO₂ (carbon dioxide) concentration acquiring step S200. In the controlling step S2102, the control unit 290 may control the display unit 210 based on a change from the CO₂ (carbon dioxide) concentration before the display unit 210 is controlled to the CO₂ (carbon dioxide) concentration after the display unit 210 is controlled.

In the controlling step S212, the control unit 290 may control the apparatus 400 used by the manipulator 310 based on the state S of the manipulator 310 judged in the judging step S208. In the controlling step S212, the control unit 290 may control the apparatus 400 based on the CO₂ (carbon dioxide) concentration acquired in the CO₂ (carbon dioxide) concentration acquiring step S200 and the reference value Vs of the CO₂ (carbon dioxide) concentration.

In the determining step S216, the judging unit 230 determines whether to acquire the CO₂ (carbon dioxide) concentration after the controlling step S212. When it is determined to acquire the CO₂ (carbon dioxide) concentration, the display method returns to the CO₂ (carbon dioxide) concentration acquiring step S200. When it is determined not to acquire the CO₂ (carbon dioxide) concentration, the display method ends.

After the apparatus 400 is controlled based on the CO₂ (carbon dioxide) concentration and the reference value Vs in the controlling step S212, the CO₂ (carbon dioxide) sensor 220 may further acquire the CO₂ (carbon dioxide) concentration in the CO₂ (carbon dioxide) concentration acquiring step S200. In the controlling step S212, the control unit 290 may control the apparatus 400 based on a change from the CO₂ (carbon dioxide) concentration before the apparatus 400 is controlled to the CO₂ (carbon dioxide) concentration after the apparatus 400 is controlled.

In the manipulation information acquiring step S204, the manipulation information acquiring unit 240 acquires the manipulation information Ip of the apparatus 400 by the manipulator 310. In the judging step S208, the judging unit 230 may judge the state S of the manipulator 310 based on the CO₂ (carbon dioxide) concentration and the manipulation information Ip. When the apparatus 400 is an exercise machine, in the displaying step S210, the display unit 210 may display a reaction to manipulation of the apparatus 400 by the manipulator 310 based on the state S of the manipulator 310 based on the CO₂ (carbon dioxide) concentration judged in the judging step 208 and the manipulation information Ip.

In the expiratory volume acquiring step S201, the expiratory volume sensor 224 further acquires the expiratory volume Vb or the expiratory cycle of the manipulator 310. In the judging step S208, the judging unit 230 may judge the state S of the manipulator 310 based on the CO₂ (carbon dioxide) concentration and the expiratory volume Vb or the expiratory cycle. In the judging step S208, the judging unit 230 may judge whether the manipulator 310 is in the anaerobic exercise state or in the aerobic exercise state based on the predetermined relationship between the CO₂ (carbon dioxide) concentration and the expiratory volume Vb or the expiratory cycle.

In the recognizing step S206, the recognizing unit 250 recognizes the manipulator 310. The relationship between the CO₂ (carbon dioxide) concentration and the expiratory volume Vb or the expiratory cycle may be predetermined for each of the plurality of manipulators 310. In the judging step S208, the judging unit 230 may judge whether the manipulator 310 recognized in the recognizing step S206 is in the anaerobic exercise state or in the aerobic exercise state based on the relationship between the CO₂ (carbon dioxide) concentration and the expiratory volume Vb or the expiratory cycle in the recognized manipulator 310. When the expiratory volume Vb is lower than the threshold Vth, in the judging step S208, the judging unit 230 may judge the emotion of the manipulator 310 based on the CO₂ (carbon dioxide) concentration.

In the biological information acquiring step S202, the biological information acquiring unit 260 acquires the biological information Ig of the manipulator 310. In the judging step S208, the judging unit 230 may judge the emotion of the manipulator 310 based on the CO₂ (carbon dioxide) concentration and the biological information Ig.

In the judging step S208, the judging unit 230 may judge an application being activated by the display device 300 and judge the state S of the manipulator 310 based on the application that is judged.

In the judging step S208, the judging unit 230 may judge a type of the application being activated by the display device 300. In the judging step S208, the judging unit 230 may judge whether the state S of the manipulator 310 based on the CO₂ (carbon dioxide) concentration acquired by the CO₂ (carbon dioxide) sensor 220 and the state of the manipulator 310 estimated based on the type of the application match. In the displaying step S210, when the state S of the manipulator 310 based on the CO₂ (carbon dioxide) concentration and the state of the manipulator 310 estimated based on the type of the application do not match, the display unit 210 may display information for prompting to quit the activated application.

In the CO₂ (carbon dioxide) concentration acquiring step S200, the one CO₂ (carbon dioxide) sensor 220-2 may acquire the CO₂ (carbon dioxide) concentration in the breath accompanied by the utterance of the manipulator 310 and the another CO₂ (carbon dioxide) sensor 220-1 may acquire the CO₂ (carbon dioxide) concentration in the breath unaccompanied by the utterance of the manipulator 310. In the judging step S208, the judging unit 230 may judge the state S of the manipulator 310 based on the CO₂ (carbon dioxide) concentration acquired by the one CO₂ (carbon dioxide) sensor 220-2 and the CO₂ (carbon dioxide) concentration acquired by the another CO₂ (carbon dioxide) sensor 220-1.

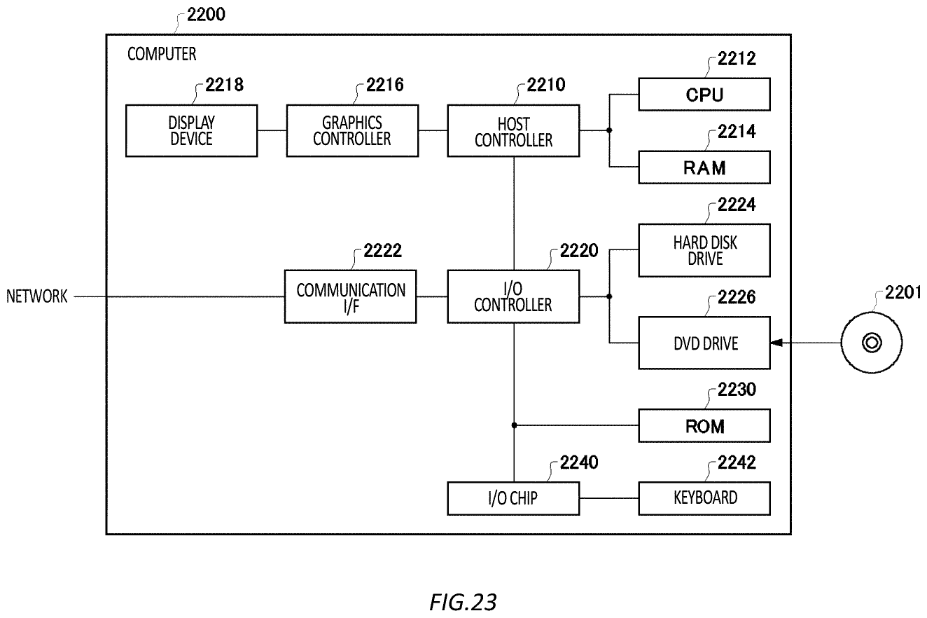

FIG. 23 is a diagram illustrating an example of a computer 2200 in which the breath sensing system 100 or the display device 300 according to one embodiment of the present invention may be entirely or partially embodied. The program installed in the computer 2200 can cause the computer 2200 to perform a manipulation associated with the breath sensing system 100 or the display device 300 according to the embodiments of the present invention or to function as one or a plurality of sections of the breath sensing system 100 or the display device 300, or to perform the manipulation or the one or plurality of sections, or can cause the computer 2200 to perform each stage according to the display method of the present invention shown in FIG. 10, FIG. 11, and FIG. 22. The program may be executed by the CPU 2212 in order to cause the computer 2200 to perform a particular manipulation associated with some or all of the blocks in the flowcharts, i.e., FIG. 10, FIG. 11, and FIG. 22, and the block diagrams, i.e., FIG. 13, FIG. 16, and FIG. 19, described in the present specification.

The computer 2200 according to one embodiment of the present invention includes the CPU 2212, a RAM 2214, a graphics controller 2216, and a display device 2218. The CPU 2212, the RAM 2214, the graphics controller 2216, and the display device 2218 are mutually connected by a host controller 2210. The computer 2200 further includes an input/output unit such as a communication interface 2222, a hard disk drive 2224, a DVD-ROM drive 2226, and an IC card drive. The communication interface 2222, the hard disk drive 2224, the DVD-ROM drive 2226, and the IC card drive, and the like are connected to the host controller 2210 via an input/output controller 2220. The computer further includes legacy input/output units such as a ROM 2230 and a keyboard 2242. The ROM 2230, the keyboard 2242, and the like are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 on a frame buffer or the like provided in the RAM 2214 or in the RAM 2214 itself to cause the image data to be displayed on the display device 2218.

The communication interface 2222 communicates with other electronic devices via a network. The hard disk drive 2224 stores programs and data used by the CPU 2212 in the computer 2200. The DVD-ROM drive 2226 reads the programs or the data from the DVD-ROM 2201, and provides the read programs or data to the hard disk drive 2224 via the RAM 2214. The IC card drive reads programs and data from an IC card, or writes programs and data to the IC card.

The ROM 2230 stores a boot program or the like executed by the computer 2200 at the time of activation, or a program depending on the hardware of the computer 2200. The input/output chip 2240 may connect various input/output units via a parallel port, a serial port, a keyboard port, a mouse port, or the like to the input/output controller 2220.

Programs are provided by a computer readable medium such as the DVD-ROM 2201 or the IC card. The programs are read from the computer readable medium, are installed in the hard disk drive 2224, the RAM 2214, or the ROM 2230 which is also an example of the computer readable medium, and are executed by the CPU 2212. The information processing described in these programs is read by the computer 2200, and provides cooperation between the programs and the various types of hardware resources. An apparatus or method may be constituted by implementing the operation or processing of information in accordance with the usage of the computer 2200.

For example, when a communication is executed between the computer 2200 and an external device, the CPU 2212 may execute a communication program loaded onto the RAM 2214 to instruct communication processing to the communication interface 2222, based on the processing described in the communication program. The communication interface 2222, under control of the CPU 2212, reads data to be sent stored on a sending buffering region provided in a recording medium such as the RAM 2214, the hard disk drive 2224, the DVD-ROM 2201, or the IC card, and sends the read data to be sent to a network or writes reception data received from a network to a reception buffering region or the like provided on the recording medium.

The CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, or the like. The CPU 2212 may execute various types of processing on the data on the RAM 2214. The CPU 2212 may then write back the processed data to the external recording medium.

Various types of information, such as various types of programs, data, tables, and databases, may be stored in the recording medium to undergo information processing. The CPU 2212 may execute various types of processing on the data read from the RAM 2214, which includes various types of operations, information processing, condition judging, conditional branch, unconditional branch, search or replacement of information, or the like, as described throughout the present disclosure and designated by an instruction sequence of programs. The CPU 2212 may write the result back to the RAM 2214.

The CPU 2212 may search for information in a file, a database, or the like in the recording medium. For example, when a plurality of entries, each having an attribute value of a first attribute associated with an attribute value of a second attribute, are stored in the recording medium, the CPU 2212 may search for an entry matching the condition whose attribute value of the first attribute is designated, from among the plurality of entries, read the attribute value of the second attribute stored in the entry, and read a second attribute value to acquire the attribute value of the second attribute associated with the first attribute satisfying the predetermined condition.

The program or software modules described above may be stored in the computer readable media on the computer 2200 or of the computer 2200. A recording medium such as a hard disk or a RAM provided in a server system connected to a dedicated communication network or the Internet can be used as the computer readable media. The program may be provided to the computer 2200 by the recording medium.

While the present invention has been described by way of the embodiments, the technical scope of the present invention is not limited to the scope described in the above-described embodiments. It is apparent to persons skilled in the art that various alterations or improvements can be made to the above-described embodiments. It is also apparent from the described scope of the claims that the embodiments added with such alterations or improvements can be included in the technical scope of the present invention.

### ITEM 1-1

A breath sensing system, including:
a main body;
a breath sensor which senses a breath, the breath sensor being provided in the main body and having two operating modes having different power consumption; and
a control unit which controls the operating modes of the breath sensor based on a state of at least one of the main body or the breath sensor.

### ITEM 1-2

The breath sensing system according to Item 1-1,
wherein the main body includes
a fixed part; and
a movable part provided to be movable relative to the fixed part and provided with the breath sensor,
wherein the control unit controls the operating modes of the breath sensor based on a state of the movable part.

### ITEM 1-3

The breath sensing system according to Item 1-2, wherein the movable part includes a sound sensing unit which senses a sound.

### ITEM 1-4

The breath sensing system according to Item 1-1, wherein
the main body includes a sound sensing unit which senses a sound,
the control unit controls the operating modes of the breath sensor based on a state of the sound sensed by the sound sensing unit.

### ITEM 1-5

The breath sensing system according to Item 1-1, wherein
the main body includes a temperature sensing unit which senses a temperature, and
the control unit controls the operating modes of the breath sensor based on the temperature sensed by the temperature sensing unit.

### ITEM 1-6

The breath sensing system according to Item 1-1, wherein
the main body includes a distance sensing unit which senses a distance from another object, and
the control unit controls the operating modes of the breath sensor based on the distance sensed by the distance sensing unit.

### ITEM 1-7

The breath sensing system according to Item 1-1, wherein
a state of the main body changes according to a control signal from application software, and
the control unit controls the operating modes of the breath sensor based on the control signal.

### ITEM 1-8

The breath sensing system according to Item 1-1, wherein
the main body includes a storage battery which supplies electrical power, and
the control unit controls the operating modes of the breath sensor based on a remaining capacity of the storage battery.

### ITEM 1-9

The breath sensing system according to Item 1-1, wherein
the main body has a plurality of operating states having different power consumption, and
the control unit controls the operating modes of the breath sensor based on an operating state of the main body.

### ITEM 1-10

The breath sensing system according to Item 1-1, wherein the control unit controls the operating modes of the breath sensor based on the breath sensed by the breath sensor.

### ITEM 1-11

The breath sensing system according to any one of Items 1-1 to 1-10, wherein
the breath sensor has, as the operating modes, an active state in which sensing of breath is enabled and a standby state in which power consumption is lower than the active state, and
the control unit controls the operating modes of the breath sensor based on a state of the main body.

### ITEM 1-12

The breath sensing system according to Item 1-11, wherein
the control unit controls the operating modes of the breath sensor by comparing a state value indicating a state of the main body and a reference value that is set, and
the reference value for transitioning the breath sensor from the active state to the standby state is different from the reference value for transitioning the breath sensor from the standby state to the active state.

### ITEM 1-13

The breath sensing system according to Item 1-11, wherein
the main body has the state of a plurality of types, and the state of each of the plurality of types being variable,
the control unit controls a state of the breath sensor based on the state of different types of the main body for when the breath sensor is transitioned from the active state to the standby state and for when the breath sensor is transitioned from the standby state to the active state.

### ITEM 1-14

The breath sensing system according to Item 1-11, wherein
the control unit adjusts a determination criterion of the state of the main body when controlling the operating modes of the breath sensor at a present time, based on the state of the main body used for determination for controlling the operating modes of the breath sensor at a previous time.

### ITEM 1-15

The breath sensing system according to any one of Items 1-1 to 1-10, wherein
the main body has the state of a plurality of types, the state of each of the plurality of types being variable, and
the control unit controls the operating modes of the breath sensor based on the state of two or more types of the main body.

### ITEM 1-16

The breath sensing system according to any one of Items 1-1 to 1-10, wherein
the breath sensor includes:
a light emitting unit which emits light toward a path through which the breath passes; and
a first light receiving unit which receives at least a part of the light emitted by the light emitting unit and outputs a first light reception signal according to a light reception result.

### ITEM 2-1

A display device, including:
a head-mounted display unit; and
a carbon dioxide sensor which acquires a carbon dioxide concentration in a breath of a manipulator of the display unit.

### ITEM 2-2

The display device according to Item 2-1, further including a judging unit which judges a state of the manipulator based on the carbon dioxide concentration,
wherein the display unit displays the state of the manipulator judged by the judging unit.

### ITEM 2-3

The display device according to Item 2-2, further including a control unit which controls the display unit based on the state of the manipulator judged by the judging unit.

### ITEM 2-4

The display device according to Item 2-3, wherein the control unit controls the display unit based on the carbon dioxide concentration and a reference value of the carbon dioxide concentration corresponding to the state of the manipulator.

### ITEM 2-5

The display device according to Item 2-4, wherein
the carbon dioxide sensor further acquires the carbon dioxide concentration after the control unit controlled the display unit based on the carbon dioxide concentration and the reference value, and
the control unit controls the display unit based on a change from the carbon dioxide concentration before the display unit is controlled to the carbon dioxide concentration after the display unit is controlled.

### ITEM 2-6

The display device according to Item 2-1, further including:
a judging unit which judges the state of the manipulator based on the carbon dioxide concentration; and
a control unit which controls an apparatus used by the manipulator based on the state of the manipulator judged by the judging unit.

### ITEM 2-7

The display device according to Item 2-6, wherein the control unit controls the apparatus based on the carbon dioxide concentration and a reference value of the carbon dioxide concentration corresponding to the state of the manipulator.

### ITEM 2-8

The display device according to Item 2-7, wherein
the carbon dioxide sensor further acquires the carbon dioxide concentration after the control unit controlled the apparatus based on the carbon dioxide concentration and the reference value, and
the control unit further controls the apparatus based on a change from the carbon dioxide concentration before the apparatus is controlled to the carbon dioxide concentration after the apparatus is controlled.

### ITEM 2-9

The display device according to Item 2-6, wherein
the apparatus is an exercise machine,
the display device further includes a manipulation information acquiring unit which acquires manipulation information of the apparatus by the manipulator, and
the display unit displays a reaction to a manipulation to the apparatus by the manipulator based on the state of the manipulator based on the carbon dioxide concentration judged by the judging unit and the manipulation information.

### ITEM 2-10

The display device according to Item 2-2 or 2-6, further including an expiratory volume sensor which acquires an expiratory volume of the manipulator,
wherein the judging unit judges the state of the manipulator based on the carbon dioxide concentration and the expiratory volume.

### ITEM 2-11

The display device according to Item 2-10, wherein the judging unit judges whether the manipulator is in an anaerobic exercise state or in an aerobic exercise state based on a predetermined relationship between the carbon dioxide concentration and the expiratory volume.

### ITEM 2-12

The display device according to Item 2-11, further including a recognizing unit which recognizes the manipulator, wherein
the manipulator includes a plurality of manipulators and the relationship is predetermined for each of the plurality of manipulators, and
the judging unit judges whether the manipulator recognized by the recognizing unit is in the anaerobic exercise state or in the aerobic exercise state based on the relationship predetermined for the manipulator recognized by the recognizing unit.

### ITEM 2-13

The display device according to Item 2-10, wherein the judging unit judges an emotion of the manipulator based on the carbon dioxide concentration when the expiratory volume is lower than a predetermined threshold.

### ITEM 2-14

The display device according to Item 2-13, further including a biological information acquiring unit which acquires biological information of the manipulator,
wherein the judging unit judges the emotion of the manipulator based on the carbon dioxide concentration and the biological information.

### ITEM 2-15

The display device according to Item 2-2 or 2-6, wherein
the judging unit further judges a type of an application while the display device is activated, and estimates the state of the manipulator based on the type of the application that is judged, and
the display unit displays information for prompting to quit the application that is activated, when the state of the manipulator based on the carbon dioxide concentration judged by the judging unit and the state of the manipulator estimated based on the type of the application do not match.

### ITEM 2-16

A display method, including:
acquiring, by a carbon dioxide sensor, a carbon dioxide concentration in a breath of a manipulator manipulating a head-mounted display unit; and
displaying, by the display unit, a state of the manipulator based on the carbon dioxide concentration.

### ITEM 2-17

The display program which causes a computer to function as the display device of any one of Items 2-1 to from 2-9.

### EXPLANATION OF REFERENCES

10: breath sensor, 11: gas sensor, 12: path, 14: light, 16: light emitting unit, 18: first light receiving unit, 20: operating unit, 22: second light receiving unit, 24: optical filter, 30: support member, 32: mirror, 40: substrate, 41: first main surface, 42: second main surface, 44: light emitting element, 100: breath sensing system, 102: main body, 110: fixed part, 111: connector, 112: sound generating unit, 113: arm part, 117: storage battery, 120: movable part, 122: sound sensing unit, 140: control unit, 150: state sensing unit, 151: positional state sensing unit, 152: environmental state sensing unit, 153: operating state sensing unit, 154: mounting sensing unit, 155: signal sensing unit, 156: remaining capacity sensing unit, 157: power consumption sensing unit, 200: user, 210: display unit, 212: housing, 214: bottom surface, 220: CO₂ (carbon dioxide) sensor, 222: aperture, 224: expiratory volume sensor, 230: judging unit, 240: manipulation information acquiring unit, 250: recognizing unit, 260: biological information acquiring unit, 270: storage unit, 280: sending unit, 290: control unit, 300: display device, 310: manipulator, 400: apparatus, 410: display unit, 420: transceiver unit, 430: control unit, 440: manipulation unit, 450: recognizing unit, 2200: computer, 2201: DVD-ROM, 2210: host controller, 2212: CPU, 2214: RAM, 2216: graphics controller, 2218: display device, 2220: input/output controller, 2222: communication interface, 2224: hard disk drive, 2226: DVD-ROM drive, 2230: ROM, 2240: input/output chip, 2242: keyboard.

What is claimed is:

1. A breath sensor which senses a breath generated by breathing, comprising:
a light emitting unit which emits light toward a path through which the breath passes;
a first light receiving unit which receives at least a part of the light emitted from the light emitting unit and outputs a first light reception signal according to a light reception result; and
an operating unit which performs an operation on the first light reception signal,
wherein the operating unit calculates a baseline of a waveform of the first light reception signal based on a frequency component of the first light reception signal lower than a first cut-off frequency that is set, and senses the breath based on a signal obtained by removing the baseline from the first light reception signal.

2. The breath sensor according to claim 1, wherein the operating unit calculates at least one of a cycle or a length of the breathing.

3. The breath sensor according to claim 1, wherein the first cut-off frequency is set according to a change in a signal value of the first light reception signal.

4. The breath sensor according to claim 1, wherein the operating unit sets the first cut-off frequency in a case where the signal value of the first light reception signal for which a value of the baseline is to be calculated is increased relative to a signal value at a past timing in a temporal waveform of the first light reception signal to be higher compared to the first cut-off frequency in a case where the signal value is decreased relative to the signal value at the past timing.

5. The breath sensor according to claim 4, wherein the operating unit sets the first cut-off frequency in a case where the signal value of the first light reception signal for which the value of the baseline is to be calculated is increased by 20% or more relative to the signal value at the past timing in the temporal waveform of the first light reception signal to be higher compared to the first cut-off frequency in a case where the signal value is decreased by 20% or more relative to the signal value at the past timing.

6. The breath sensor according to claim 4, wherein the operating unit sets the first cut-off frequency in a case where the signal value of the first light reception signal for which the value of the baseline is to be calculated is increased relative to a signal value at an immediately preceding timing in the temporal waveform of the first light reception signal to be higher by ten times or more compared to the first cut-off frequency in a case where the signal value is decreased relative to the signal value at the immediately preceding timing.

7. The breath sensor according to claim 6, wherein the operating unit sets the first cut-off frequency in a case where the signal value of the first light reception signal for which the value of the baseline is to be calculated is increased by 20% or more relative to the signal value at the immediately preceding timing in the temporal waveform of the first light reception signal to be higher by ten times or more compared to the first cut-off frequency in a case where the signal value is decreased by 20% or more relative to the signal value at the immediately preceding timing.

8. The breath sensor according to claim 6, wherein the first cut-off frequency is 3 Hz or less.

9. The breath sensor according to claim 1, further comprising a second light receiving unit which receives light, included in the light emitted by the light emitting unit, which did not pass through the path through which the breath passes, and outputs a second light reception signal according to a light reception result,
wherein the operating unit corrects the first light reception signal based on a signal value of the second light reception signal.

10. The breath sensor according to claim 9, wherein
the operating unit generates the first light reception signal that is corrected by using a frequency component of the second light reception signal lower than a second cut-off frequency that is set and a frequency component of the first light reception signal lower than a third cut-off frequency that is set, and
the second cut-off frequency is lower than the third cut-off frequency.

11. The breath sensor according to claim 6, further comprising an optical filter which is located between the light emitting unit and the first light receiving unit on a light path of the light and which restricts a wavelength of the light that enters the first light receiving unit.

12. A gas sensor which senses a gas to be sensed, comprising:
a light emitting unit which emits light toward a path through which the gas to be sensed passes;
a first light receiving unit which receives at least a part of the light emitted by the light emitting unit and outputs a first light reception signal according to a light reception result; and
an operating unit which performs an operation on the first light reception signal,
wherein the operating unit calculates a baseline of a waveform of the first light reception signal based on a frequency component of the first light reception signal lower than a first cut-off frequency that is set, and senses the gas to be sensed based on a signal obtained by removing the baseline from the first light reception signal.

13. A breath sensing method for sensing a breath generated by breathing, comprising:
emitting, by a light emitting unit, light toward a path through which the breath passes;
firstly receiving, by a first light receiving unit, at least a part of the light emitted in the emitting the light;
firstly outputting, by the first light receiving unit, a first light reception signal according to a light reception result in the firstly receiving the light; and
performing, by an operating unit, an operation on the first light reception signal output in the firstly outputting,
wherein the performing the operation includes:
calculating, by the operating unit, a baseline of a waveform of the first light reception signal based on a frequency component of the first light reception signal lower than a first cut-off frequency that is set; and
sensing, by the operating unit, the breath based on a signal obtained by removing the baseline calculated in the calculating from the first light reception signal.

14. The breath sensing method according to claim 13, wherein, in the performing the operation, the operating unit calculates at least one of a cycle or a length of the breathing.

15. The breath sensing method according to claim 13, wherein the first cut-off frequency is set according to a change in a signal value of the first light reception signal.

16. The breath sensing method according to claim 13, wherein, in the performing the operation, the operating unit sets the first cut-off frequency in a case where the signal value of the first light reception signal for which a value of the baseline is to be calculated is increased relative to a signal value at a past timing in a temporal waveform of the first light reception signal to be higher compared to the first cut-off frequency in a case where the signal value is decreased relative to the signal value at the past timing.

17. The breath sensing method according to claim 16, wherein, in the performing the operation, the operating unit sets the first cut-off frequency in a case where the signal value of the first light reception signal for which the value of the baseline is to be calculated is increased by 20% or more relative to the signal value at the past timing in the temporal waveform of the first light reception signal to be higher compared to the first cut-off frequency in a case where the signal value is decreased by 20% or more relative to the signal value at the past timing.

18. The breath sensing method according to claim 16, wherein, in the performing the operation, the operating unit sets the first cut-off frequency in a case where the signal value of the first light reception signal for which the value of the baseline is to be calculated is increased relative to the signal value at an immediately preceding timing in the temporal waveform of the first light reception signal to be higher by ten times or more compared to the first cut-off frequency in a case where the signal value is decreased relative to the signal value at the immediately preceding timing.

19. The breath sensing method according to claim 18, wherein, in the performing the operation, the operating unit sets the first cut-off frequency in a case where the signal value of the first light reception signal for which the value of the baseline is to be calculated is increased by 20% or more relative to a signal value at an immediately preceding timing in the temporal waveform of the first light reception signal to be higher by ten times or more compared to the first cut-off frequency in a case where the signal value is decreased by 20% or more relative to the signal value at the immediately preceding timing.

20. The breath sensing method according to claim 13, further comprising:
secondly receiving, by a second light receiving unit, light, included in the light emitted by the light emitting unit, which did not pass through the path through which the breath passes; and
secondly outputting, by the second light receiving unit, a second light reception signal according to a light reception result in the secondly receiving the light,
wherein the performing the operation further includes correcting, by the operating unit, the first light reception signal based on a signal value of the second light reception signal.
